(19) United States
(12) Patent Application Publication
(10) Pub. No.: US 2025/0059170 A1
(43) Pub. Date: Feb. 20, 2025

(54) SOLID STATE FORMS OF AN FGFR INHIBITOR

(71) Applicant: Kinnate Biopharma Inc., San Diego, CA (US)

(72) Inventors: Stephen W. KALDOR, San Diego, CA (US); Toufike KANOUNI, Palm Beach Gardens, FL (US); Andrew PHIMISTER, Kensington, CA (US); Jayachandra P. REDDY, West Palm Beach, FL (US); John TYHONAS, San Diego, CA (US); Shubham CHOPADE, Madison, AL (US); Preetanshu PANDEY, San Diego, CA (US); Jason M. COX, Rancho Santa Fe, CA (US); Robert KANIA, Del Mar, CA (US)

(21) Appl. No.: 18/717,390

(22) PCT Filed: Dec. 7, 2022
(86) PCT No.: PCT/US2022/081060

§ 371 Date: Jun. 6, 2024

Related U.S. Application Data
(60) Provisional application No. 63/287,212, filed on Dec. 8, 2021.

(51) Int. Cl. C07D 403/14

(52) U.S. Cl. CPC C07D 403/14

(57) ABSTRACT

The present disclosure relates to solid state forms of 1-((3S,5R)-1-acryloyl-5-(methoxymethyl)pyrrolidin-3-yl)-3-((1-cyclopropyl-4, 6-difluoro-1H-benzo[d]imidazol-5-yl)ethynyl)-5-(methylamino)-1H-pyrazole-4-carboxamide. Such solid state forms are useful in preparation of pharmaceutical compositions and dosage forms for the treatment of disease.

### CROSS-REFERENCE TO RELATED APPLICATION

This application claims the benefit of U.S. Patent Application No. 63/287,212, filed on Dec. 8, 2021, which is hereby incorporated by reference in its entirety.

### BACKGROUND

Fibroblast growth factor receptors (FGFRs) are a subfamily of receptor tyrosine kinases (RTKs) that bind to members of the fibroblast growth factor family of proteins. Deregulation of the fibroblast growth factor/FGF receptor network occurs frequently in tumors. Accordingly, therapies that target aberrant FGFR kinase activity are desired for use in the treatment of cancer and other disorders. One such modulator of FGFR kinase is 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

### SUMMARY

The present disclosure relates to solid state forms of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, herein after known as Compound 1. The molecular structure of Compound 1 is shown below:

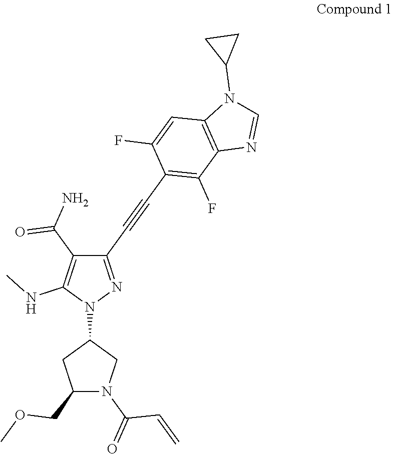 [SMILES: C=CC(=O)N1C[C@@H](n2nc(C#Cc3c(F)cc4c(ncn4C4CC4)c3F)c(C(N)=O)c2NC)C[C@@H]1COC]

1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide

Disclosed herein is a solid form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1).

Disclosed herein is a solid form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the solid form is crystalline.

Disclosed herein is a solid crystalline form of Compound 1 wherein the solid form is crystalline. In some embodiments, the solid form exhibits an X-ray powder diffraction reflection at a 2-theta value of 11.8°±0.3. In some embodiments, the solid form exhibits an X-ray powder diffraction reflection at a 2-theta value of 7.9°±0.3 and 13.7°±0.3. In some embodiments, the solid form exhibits an X-ray powder diffraction reflection at a 2-theta value of 9.5°±0.3, 18.9°±0.3, and 26.8°±0.3. In some embodiments, the solid form exhibits an X-ray powder diffraction reflection at a 2-theta value of 15.9°±0.3, 20.5°±0.3, and 22.3°±0.3. In some embodiments, the solid form exhibits an X-ray powder diffraction reflection at a 2-theta value of 20.8°±0.3, 25.7°±0.3, and 26.3°±0.3. In some embodiments, the solid form exhibits at least one X-ray powder diffraction reflection selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits at least two X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits at least three X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits at least four X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits at least five X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits at least six X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the solid form exhibits the X-ray powder diffraction pattern as shown in FIG. 4. In some embodiments, the solid form exhibits a differential scanning calorimetry thermogram comprising an endothermic peak at 193.1° C.±5.0. In some embodiments, the solid form exhibits the differential scanning calorimetry thermogram as shown in FIG. 5. In some embodiments, the solid form exhibits less than 1.1%±0.5 weight loss up to 200° C.±10.0 as determined by thermogravimetric analysis. In some embodiments, the solid form exhibits the thermogravimetric analysis thermogram as shown in FIG. 5. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 8.7°±0.3. In some embodiments, the solid form exhibits an X-ray powder diffraction reflection at a 2-theta value of 5.0°±0.3 and 12.8°±0.3. In some embodiments, the solid form exhibits an X-ray powder diffraction reflection at a 2-theta value of 23.9°±0.3 and 24.3°±0.3. In some embodiments, the solid form exhibits an X-ray powder diffraction reflection at a 2-theta value of 11.5°±0.3, 20.3°±0.3, and 21.2°±0.3. In some embodiments, the solid form exhibits an X-ray powder diffraction reflection at a 2-theta value of 11.6°±0.3, 15.9°±0.3, and 25.8°±0.3. In some embodiments, the solid form exhibits at least one X-ray powder diffraction reflection selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits at least two X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits at least three X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits at least four X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits at least five X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits at least six X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the solid form exhibits the X-ray powder diffraction pattern as shown in FIG. 7. In some embodiments, the solid form exhibits a differential scanning calorimetry thermogram comprising endothermic peaks at 164.0° C.±5.0 and 190.6° C.±5.0. In some embodiments, the solid form exhibits the differential scanning calorimetry thermogram as shown in FIG. 8. In some embodiments, the solid form exhibits less than 2.9%±0.5 weight loss up to 205° C.±10.0 as determined by thermogravimetric analysis. In some embodiments, the solid form exhibits the thermogravimetric analysis thermogram as shown in FIG. 8. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 5.1°±0.3. In some embodiments, the solid form exhibits an X-ray powder diffraction reflection at a 2-theta value of 10.2°±0.3 and 8.6°±0.3. In some embodiments, the solid form exhibits an X-ray powder diffraction reflection at a 2-theta value of 12.2°±0.3 and 17.3°±0.3. In some embodiments, the solid form exhibits an X-ray powder diffraction reflection at a 2-theta value of 6.4°±0.3, 13.4°±0.3, and 15.3°±0.3. In some embodiments, the solid form exhibits an X-ray powder diffraction reflection at a 2-theta value of 15.8°±0.3, and 26.0°±0.3. In some embodiments, the solid form exhibits at least one X-ray powder diffraction reflection selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits at least two X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits at least three X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits at least four X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits at least five X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits at least six X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the solid form exhibits the X-ray powder diffraction pattern as shown in FIG. 9. In some embodiments, the solid form exhibits a differential scanning calorimetry thermogram comprising endothermic peaks at 66.2° C.±5.0, 106.4° C.±5.0, and 193.6° C.±5.0. In some embodiments, the solid form exhibits the differential scanning calorimetry thermogram as shown in FIG. 10. In some embodiments, the solid form exhibits less than 2.9%±0.5 weight loss up to 94° C.±10.0 as determined by thermogravimetric analysis. In some embodiments, the solid form exhibits the thermogravimetric analysis thermogram as shown in FIG. 10. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 5.1°±0.3. In some embodiments, the solid form exhibits an X-ray powder diffraction reflection at a 2-theta value of 10.4°±0.3. In some embodiments, the solid form exhibits an X-ray powder diffraction reflection at a 2-theta value of 6.4°±0.3 and 25.6°±0.3. In some embodiments, the solid form exhibits an X-ray powder diffraction reflection at a 2-theta value of 8.7°±0.3, 11.9°±0.3, and 15.6°±0.3. In some embodiments, the solid form exhibits an X-ray powder diffraction reflection at a 2-theta value of 13.5°±0.3, and 25.8°±0.3. In some embodiments, the solid form exhibits at least one X-ray powder diffraction reflection selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits at least two X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits at least three X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits at least four X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits at least five X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits at least six X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the solid form exhibits the X-ray powder diffraction pattern as shown in FIG. 11. In some embodiments, the solid form exhibits a differential scanning calorimetry thermogram comprising an endothermic peak at 105.8° C.±5.0. In some embodiments, the solid form exhibits the differential scanning calorimetry thermogram as shown in FIG. 12. In some embodiments, the solid form exhibits less than 2.1%±0.5 weight loss up to 101° C.±10.0 as determined by thermogravimetric analysis. In some embodiments, the solid form exhibits the thermogravimetric analysis thermogram as shown in FIG. 12. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 9.5°±0.3. In some embodiments, the solid form exhibits an X-ray powder diffraction reflection at a 2-theta value of 5.2°±0.3 and 23.7°±0.3. In some embodiments, the solid form exhibits an X-ray powder diffraction reflection at a 2-theta value of 5.7°±0.3, 10.2°±0.3, and 19.5°±0.3. In some embodiments, the solid form exhibits an X-ray powder diffraction reflection at a 2-theta value of 6.5°±0.3, 15.5°±0.3, and 26.8°±0.3. In some embodiments, the solid form exhibits an X-ray powder diffraction reflection at a 2-theta value of 7.4°±0.3, 15.0°±0.3, 19.0°±0.3, and 24.7°±0.3. In some embodiments, the solid form exhibits at least one X-ray powder diffraction reflection selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits at least two X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits at least three X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits at least four X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits at least five X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits at least six X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the solid form exhibits the X-ray powder diffraction pattern as shown in FIG. 13. In some embodiments, the solid form exhibits a differential scanning calorimetry thermogram comprising endothermic peaks at 112.3° C.±5.0 and 193.7° C.±5.0. In some embodiments, the solid form exhibits the differential scanning calorimetry thermogram as shown in FIG. 14. In some embodiments, the solid form exhibits less than 4.3%±0.5 weight loss up to 126° C.±10.0 as determined by thermogravimetric analysis. In some embodiments, the solid form exhibits the thermogravimetric analysis thermogram as shown in FIG. 14. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 5.4°±0.3. In some embodiments, the solid form exhibits an X-ray powder diffraction reflection at a 2-theta value of 9.8°±0.3 and 23.4°±0.3. In some embodiments, the solid form exhibits an X-ray powder diffraction reflection at a 2-theta value of 5.0°±0.3 and 15.2°±0.3. In some embodiments, the solid form exhibits an X-ray powder diffraction reflection at a 2-theta value of 10.1°±0.3, 13.6°±0.3, and 26.7°±0.3. In some embodiments, the solid form exhibits an X-ray powder diffraction reflection at a 2-theta value of 9.1°±0.3, 25.6°±0.3, and 27.2°±0.3. In some embodiments, the solid form exhibits at least one X-ray powder diffraction reflection selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits at least two X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits at least three X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits at least four X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits at least five X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits at least six X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the solid form exhibits the X-ray powder diffraction pattern as shown in FIG. 15. In some embodiments, the solid form exhibits a differential scanning calorimetry thermogram comprising endothermic peaks at 120.8° C.±5.0 and 195.1° C.±5.0. In some embodiments, the solid form exhibits the differential scanning calorimetry thermogram as shown in FIG. 16. In some embodiments, the solid form exhibits less than 3.9%±0.5 weight loss up to 119° C.±10.0 as determined by thermogravimetric analysis. In some embodiments, the solid form exhibits the thermogravimetric analysis thermogram as shown in FIG. 16. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 5.8°±0.3. In some embodiments, the solid form exhibits an X-ray powder diffraction reflection at a 2-theta value of 5.2°±0.3 and 15.2°±0.3. In some embodiments, the solid form exhibits an X-ray powder diffraction reflection at a 2-theta value of 5.0°±0.3 and 10.1±0.3. In some embodiments, the solid form exhibits an X-ray powder diffraction reflection at a 2-theta value of 8.4°±0.3, 9.8°±0.3, and 23.4°±0.3. In some embodiments, the solid form exhibits an X-ray powder diffraction reflection at a 2-theta value of 10.4°±0.3, 14.2°±0.3, and 25.5°±0.3. In some embodiments, the solid form exhibits at least one X-ray powder diffraction reflection selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits at least two X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits at least three X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits at least four X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits at least five X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits at least six X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the solid form exhibits the X-ray powder diffraction pattern as shown in FIG. 17. In some embodiments, the solid form exhibits a differential scanning calorimetry thermogram comprising endothermic peaks at 108.7° C.±5.0 and 195.1° C.±5.0. In some embodiments, the solid form exhibits the differential scanning calorimetry thermogram as shown in FIG. 18. In some embodiments, the solid form exhibits less than 7.4%±0.5 weight loss up to 140° C.±10.0 as determined by thermogravimetric analysis. In some embodiments, the solid form exhibits the thermogravimetric analysis thermogram as shown in FIG. 18. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is crystalline. In some embodiments, the solid form exhibits an X-ray powder diffraction reflection at a 2-theta value of 12.3°±0.3. In some embodiments, the solid form exhibits an X-ray powder diffraction reflection at a 2-theta value of 6.1°±0.3 and 13.4°±0.3. In some embodiments, the solid form exhibits an X-ray powder diffraction reflection at a 2-theta value of 7.9°±0.3 and 10.5°±0.3. In some embodiments, the solid form exhibits an X-ray powder diffraction reflection at a 2-theta value of 11.8°±0.3, 25.0°±0.3, and 25.7°±0.3. In some embodiments, the solid form exhibits an X-ray powder diffraction reflection at a 2-theta value of 18.6°±0.3, 23.0°±0.3, and 25.2°±0.3. In some embodiments, the solid form exhibits at least one X-ray powder diffraction reflection selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits at least two X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits at least three X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits at least four X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits at least five X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits at least six X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the solid form exhibits the X-ray powder diffraction pattern as shown in FIG. 19. In some embodiments, the solid form exhibits a differential scanning calorimetry thermogram comprising an endothermic peak at 179.3° C.±5.0. In some embodiments, the solid form exhibits the differential scanning calorimetry thermogram as shown in FIG. 20. In some embodiments, the solid form exhibits less than 2.9%±0.5 weight loss up to 245° C.±10.0 as determined by thermogravimetric analysis. In some embodiments, the solid form exhibits the thermogravimetric analysis thermogram as shown in FIG. 20. In some embodiments, the solid form is substantially free of other crystalline or amorphous forms. In some embodiments, the amount of other crystalline or amorphous forms is 5% (w/w) or less. In some embodiments, the solid form is substantially free of impurities. In some embodiments, the amount of impurities is 2% or less.

Disclosed herein is a solid crystalline form of Compound 1 wherein the solid form is amorphous. In some embodiments, the solid form exhibits the X-ray powder diffraction pattern as shown in FIG. 1.

Disclosed herein is a pharmaceutical composition comprising the solid form of any one of the solid forms provided herein and a pharmaceutically acceptable excipient. In some embodiments, the pharmaceutical composition further comprises a disintegrating agent.

Disclosed herein is a method of treating cancer in a patient in need thereof, comprising administering to the patient a composition comprising the solid form of any one of the solid forms provided herein and at least one pharmaceutically acceptable excipient.

Disclosed herein is a method of treating cancer in a patient in need thereof, comprising administering to the patient the solid form of any of the solid forms provided herein.

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows an X-ray diffraction pattern of amorphous Compound 1;

FIG. 2 shows an X-ray diffraction pattern of crystalline Form I of Compound 1;

FIG. 3 shows a differential scanning calorimetry and thermogravimetric analysis of Form I of Compound 1;

FIG. 4 shows an X-ray diffraction pattern of crystalline Form II of Compound 1;

FIG. 5 shows a Differential scanning calorimetry and thermogravimetric analysis of Form II of Compound 1;

FIG. 6 shows a Dynamic Vapor Sorption isotherm plot and mass plot of crystalline Form II of Compound 1;

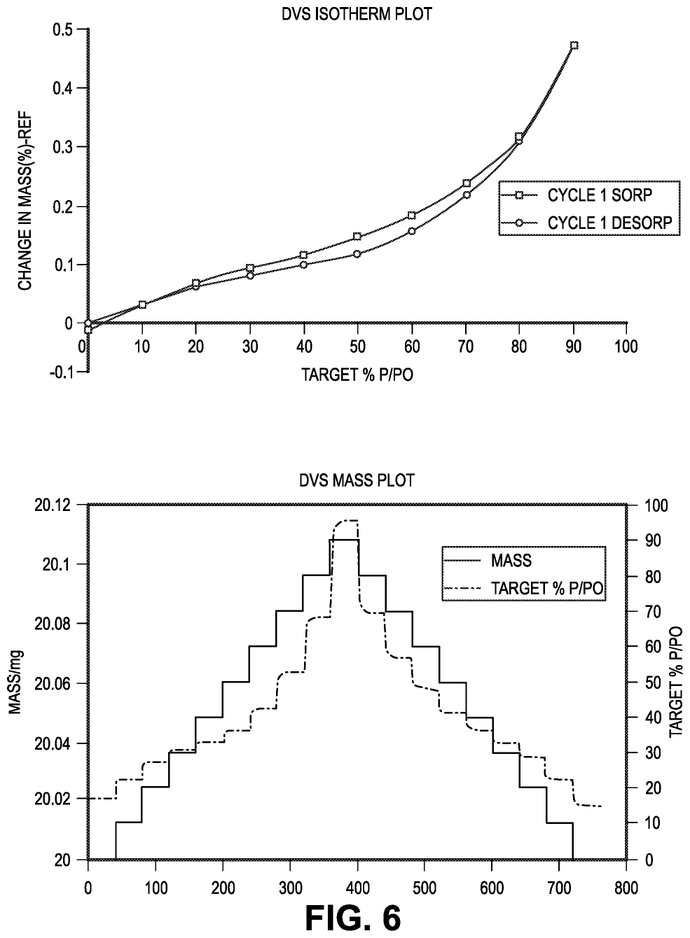

FIG. 7 shows an X-ray diffraction pattern of crystalline Form III of Compound 1;

FIG. 8 shows a differential scanning calorimetry and thermogravimetric analysis of Form III of Compound 1;

FIG. 9 shows an X-ray diffraction pattern of crystalline Form IV of Compound 1;

FIG. 10 shows a differential scanning calorimetry and thermogravimetric analysis of Form IV of Compound 1;

FIG. 11 shows an X-ray diffraction pattern of crystalline Form V of Compound 1;

FIG. 12 shows a differential scanning calorimetry and thermogravimetric analysis of Form V of Compound 1;

FIG. 13 shows an X-ray diffraction pattern of crystalline Form VI of Compound 1;

FIG. 14 shows a differential scanning calorimetry and thermogravimetric analysis of Form VI of Compound 1;

FIG. 15 shows an X-ray diffraction pattern of crystalline Form VII of Compound 1;

FIG. 16 shows a differential scanning calorimetry and thermogravimetric analysis of Form VII of Compound 1;

FIG. 17 shows an X-ray diffraction pattern of crystalline Form VIII of Compound 1;

FIG. 18 shows a differential scanning calorimetry and thermogravimetric analysis of Form VIII of Compound 1;

FIG. 19 shows an X-ray diffraction pattern of crystalline Form IX of Compound 1;

FIG. 20 shows a differential scanning calorimetry and thermogravimetric analysis of Form IX of Compound 1;

FIG. 21 shows a conversion map for select solid state forms of Compound 1; and

FIG. 22 shows an exemplary tablet manufacturing process for pharmaceutical formulations with a solid form of Compound 1.

### DETAILED DESCRIPTION OF THE INVENTION

Provided herein are ten solid state forms of Compound 1. The solid state forms of Compound 1 are identified as an amorphous form and nine crystalline forms designated as Form I, Form II, Form III, Form IV, Form V, Form VI, Form VII, Form VIII, and Form IX. In some embodiments, disclosed herein are pharmaceutical compositions comprising a solid state form of Compound 1. In some embodiments, disclosed herein are pharmaceutical compositions comprising the amorphous form, Form I, Form II, Form III, Form IV, Form V, Form VI, Form VII, Form VIII, Form IX, or any combinations thereof.

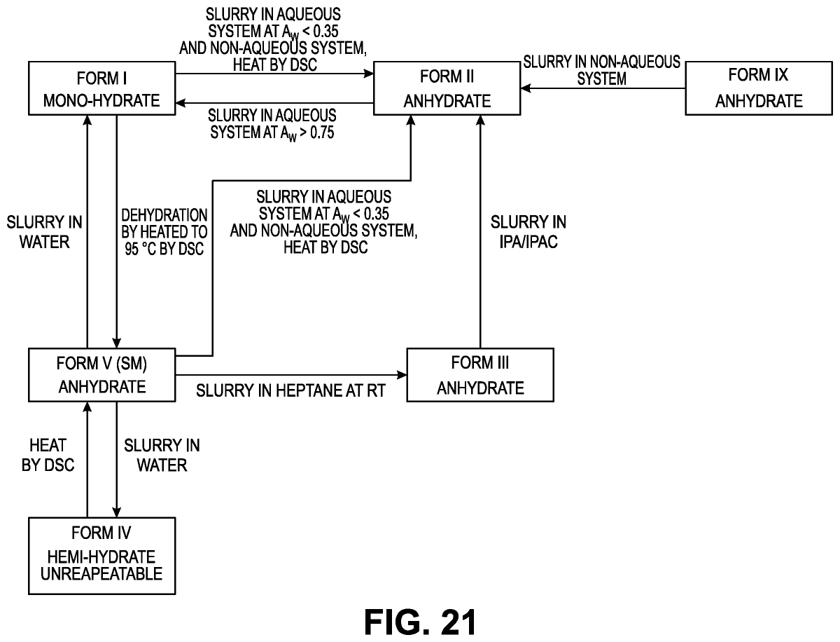

Nine crystal forms were identified, assigned as Form I-IX, including a mono-hydrate (Form I) four anhydrates (Form II, III, V and IX), a hemihydrate, (Form IV) and three solvates (Form VI, VII and VIII). The characterization data of these forms are presented in Table 15. Inter-conversion studies suggested Form II is the most stable form in non-aqueous solvents and physically stable at aw≤0.35 for RT and aw≤0.55 for 50° C. Form I also showed superior stability as compared to other forms at aw≥0.75 for 50° C. and aw≥0.55 for RT. The three solvates (Form VI, VII, and VIII) converted to Form II after desolvation and recrystallization during heating by DSC. A Conversion Map of different forms is shown in FIG. 21. Form II was evaluated by DVS, solubility and stability testing. DVS results indicated it was slightly hygroscopic with 0.47% of water uptake at 90% RH. The solubility results showed it had the highest solubility in SGF (0.8 mg/mL) and low solubility in water (about 3 μg/mL). The solubility in FeSSIF was about thirty times higher than that in FaSSIF at 24 h. Form II was physically stable at RT/92.5% RH for 2 days, physically and chemically stable at 40° C./75% RH for 6 days. The purity decreased by about 0.3% at 60° C. for 3 days and 7 days, mainly due to the increase of impurities at RRT 0.9, 0.91 and 1.07.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “hydrate” and “solvate” are meant to describe crystalline Compound 1 forms that include an amount of water or solvent, as supported by data derived from differential scanning calorimetry (DSC) experiments, thermogravimetric analysis (TGA) experiments, X-ray diffraction experiments, and/or the procedure for generating the solid crystalline form. In some embodiments, a solvate crystalline form or hydrate crystalline form comprises at least 1.5%, 1.75%, 2.0%, 2.5%, 3.0%, 4.0%, 5.0%, 6.0%, 7.0%, 8.0%, 9.0%, 10.0%, 15.0%, or 20.0% of the total weight of the sample as water, solvent, or a combination thereof, as determined by TGA. In some embodiments, a solvate crystalline form or hydrate crystalline form exhibits at least one DSC endotherm onset before or within 30° C. of the boiling point of water or the solvent(s) used in the generation of the crystalline form. For example, a hydrate crystalline form may have a DSC endotherm onset at 108° C., with the endotherm peak positioned at 124° C.

Crystalline solid forms termed a “solvate,” or “hydrate” are not meant to be limiting. For example, a solvate or hydrate can comprise a combination of water and solvent in the crystalline solid form.

The term “type,” “form,” and “pattern” are meant to be used interchangeably and are meant to refer to a particular crystalline material with properties described herein. For example, “crystalline Type A,” “crystalline Form A,” and “XRPD Pattern A” refer to the same crystalline matter. Roman numerals and integers are also meant to be interchangeable, for example “Form 1” and “Form I” are equivalent.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “substantially similar” as used herein means an analytical spectrum, such as XRPD pattern, DSC thermogram, or TGA thermogram, which resembles the reference spectrum to a great degree in both the peak locations and peak intensity.

### Characterization of Compounds and Solid State Forms

In one embodiment, the present invention provides solid state forms of Compound 1. In one embodiment, the crystalline forms are characterized by the interlattice plane intervals determined by an X-ray powder diffraction (XRPD) diffractogram. The diffractogram is typically represented by a diagram plotting the intensity of the peaks versus the location of the peaks, i.e., diffraction angle 20 (two-theta) in degrees. The characteristic peaks of a given compound can be selected according to the peak locations and their relative intensity to distinguish compounds and crystalline structures from others. Peak tables are provided with corresponding d-spacings to aid in conversion of 2-theta measurements across X-ray radiation sources (e.g. copper and molybdenum). Amorphous solid state forms were also characterized by XRPD. Amorphous solid state forms exhibit an absence of interlattice plane intervals.

Those skilled in the art recognize that the measurements of the x-ray diffraction (XRD) peak locations and/or intensity for a given crystalline form of the same compound will vary within a margin of error. The values of degree 2Θ allow appropriate error margins. Typically, the error margins are represented by “±”. For example, the degree 2Θ of “8.716°±0.3” denotes a range from 8.716°+0.3, i.e., 9.016°, to 8.716°−0.3, i.e., 8.416°. Depending on the sample preparation techniques, the calibration techniques applied to the instruments, human operational variation, and collection temperature, those skilled in the art recognize that the margin of error for an XRD can be ±0.5; ±0.4; ±0.3; ±0.2; ±0.1; ±0.05; or less. Additional details of the methods and equipment used for the XRD analysis are described in the Examples section.

In one embodiment, the crystalline forms are characterized by Differential Scanning calorimetry (DSC) and Thermogravimetric Analysis (TGA). The DSC thermogram is typically expressed by a diagram plotting the normalized heat flow in units of Watts/gram (“W/g”) versus the measured sample temperature in degree C. The DSC thermogram is generally evaluated for extrapolated onset and end (outset) temperatures, peak temperature, and heat of fusion. The single maximum value of a DSV thermogram is often used as the characteristic peak to distinguish one crystalline form from another crystalline form. The TGA thermogram is typically expressed by a diagram plotting the weight loss percentage (%) versus the measured sample temperature in degree C. In the figures disclosed herein, DSC and TGA thermograms have been plotted sharing an X axis (temperature), but have distinct Y axes of weight % and heat flow corresponding respectively to TGA and DSC measurements.

Those skilled in the art recognize that the measurements of the DSC and TGA thermograms for a given crystalline form of the same compound will vary within a margin of error. The values of a single maximum value, expressed in degree C., allow appropriate error margins. Typically, the error margins are represented by “±”. For example, the single maximum value of “53.1° C.±10.0” denotes a range from 53.1° C.±10.0, i.e., 63.1° C., to about 53.1° C.-10.0, i.e., 43.1° C. Depending on the sample preparation techniques, crystallization conditions, calibration techniques applied to the instruments, human operational variations, and etc., those skilled in the art recognize that the appropriate margin of error for a single maximum value can be ±10.0; ±7.5; ±5.0; ±2.5; ±2; ±1.5; ±1; ±0.5; or less for any of the powder diffraction reflections described herein.

Additional details of the methods and equipment used for the DSC and TGA thermogram analysis are described in the Examples section.

### Crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form I is characterized by exhibits an X-ray powder diffraction reflection at a 2-theta value of 5.0°±0.3. In some embodiments, the crystalline form exhibits an X-ray powder diffraction reflection at a 2-theta value of 10.1°±0.3 and 17.1°±0.3. In some embodiments, the crystalline form exhibits an X-ray powder diffraction reflection at a 2-theta value of 8.5°±0.3, 24.8°±0.3, and 25.8°±0.3. In some embodiments, the crystalline form exhibits an X-ray powder diffraction reflection at a 2-theta value of 9.3°±0.3, 13.0°±0.3, and 16.4°±0.3. In some embodiments, the crystalline form exhibits an X-ray powder diffraction reflection at a 2-theta value of 12.1°±0.3 and 15.2°±0.3.

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form I is characterized by at least one X-ray diffraction pattern reflection selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3. In some embodiments, the crystalline Form I is characterized by at least two X-ray diffraction pattern reflections selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3. In some embodiments, the crystalline Form I is characterized by at least three X-ray diffraction pattern reflections selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3. In some embodiments, the crystalline Form I is characterized by at least four X-ray diffraction pattern reflections selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3. In some embodiments, the crystalline Form I is characterized by at least five X-ray diffraction pattern reflections selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3. In some embodiments, the crystalline Form I is characterized by at least six X-ray diffraction pattern reflections selected from a 2 theta value of 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

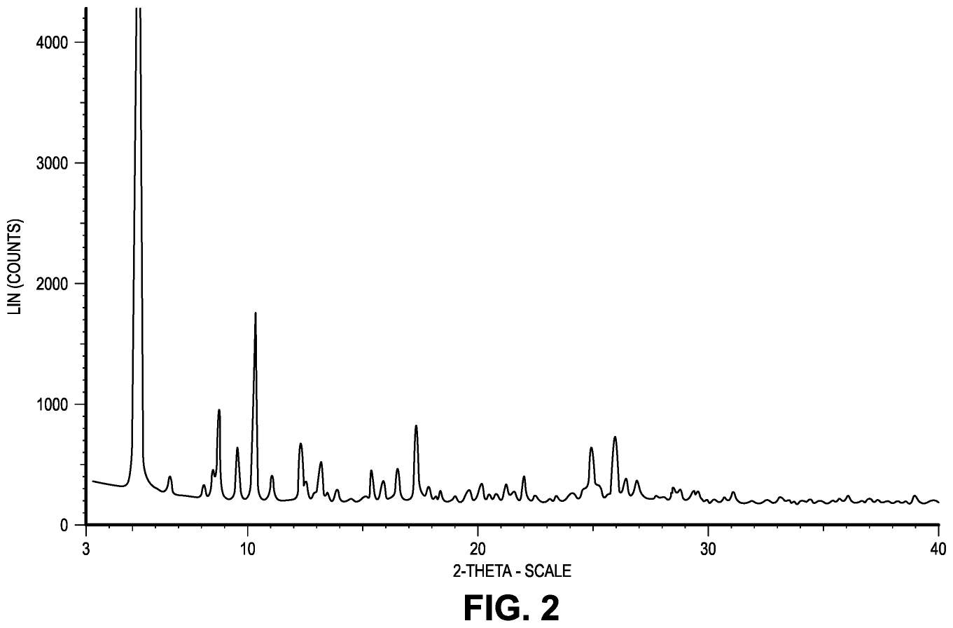

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 2.

TABLE 1
Peak listing for the X-ray powder diffractogram
of the crystalline solid state form of Form I.

| | | Rel. |
|---|---|---|
| Angle | d Value | Intensity |
| 5.015° | 17.60533 Å | 100.0% |
| 6.372° | 13.85906 Å | 2.0% |
| 7.866° | 11.23075 Å | 1.6% |
| 8.267° | 10.68709 Å | 3.3% |
| 8.516° | 10.37495 Å | 10.9% |
| 9.349° | 9.45255 Å | 6.3% |
| 10.087° | 8.76175 Å | 22.8% |
| 10.838° | 8.15650 Å | 3.1% |
| 12.107° | 7.30470 Å | 7.1% |
| 12.318° | 7.17967 Å | 2.3% |
| 12.707° | 6.96072 Å | 1.1% |
| 12.945° | 6.83316 Å | 4.7% |
| 13.244° | 6.67991 Å | 1.0% |
| 13.680° | 6.46771 Å | 1.4% |
| 14.291° | 6.19248 Å | 0.3% |
| 14.912° | 5.93602 Å | 0.5% |
| 15.184° | 5.83054 Å | 3.7% |
| 15.683° | 5.64595 Å | 2.3% |
| 16.326° | 5.42510 Å | 3.8% |
| 17.142° | 5.16865 Å | 9.0% |
| 17.665° | 5.01667 Å | 1.7% |
| 18.192° | 4.87246 Å | 1.2% |
| 18.824° | 4.71038 Å | 0.7% |
| 19.407° | 4.57026 Å | 1.0% |
| 19.972° | 4.44210 Å | 2.1% |
| 20.310° | 4.36905 Å | 0.6% |
| 20.587° | 4.31088 Å | 0.8% |
| 21.041° | 4.21875 Å | 2.0% |
| 21.371° | 4.15441 Å | 1.2% |
| 21.828° | 4.06843 Å | 3.1% |
| 22.332° | 3.97767 Å | 0.7% |
| 22.966° | 3.86927 Å | 0.4% |
| 23.232° | 3.82561 Å | 0.6% |
| 23.961° | 3.71090 Å | 0.9% |
| 24.476° | 3.63389 Å | 1.2% |
| 24.784° | 3.58955 Å | 6.5% |
| 25.055° | 3.55124 Å | 1.5% |
| 25.793° | 3.45133 Å | 7.9% |
| 26.268° | 3.38999 Å | 2.5% |
| 26.752° | 3.32978 Å | 2.3% |
| 27.830° | 3.20311 Å | 0.3% |
| 28.373° | 3.14308 Å | 1.5% |
| 28.598° | 3.11889 Å | 1.3% |
| 29.237° | 3.05213 Å | 1.1% |
| 29.425° | 3.03309 Å | 1.3% |
| 30.141° | 2.96257 Å | 0.3% |
| 30.598° | 2.91940 Å | 0.5% |
| 30.983° | 2.88394 Å | 1.3% |
| 31.764° | 2.81487 Å | 0.4% |
| 32.447° | 2.75713 Å | 0.2% |
| 33.013° | 2.71113 Å | 0.7% |
| 34.275° | 2.61417 Å | 0.3% |
| 34.753° | 2.57925 Å | 0.2% |
| 35.576° | 2.52148 Å | 0.4% |
| 35.977° | 2.49428 Å | 0.9% |
| 36.563° | 2.45566 Å | 0.2% |
| 36.933° | 2.43188 Å | 0.4% |
| 37.235° | 2.41285 Å | 0.3% |
| 38.884° | 2.31425 Å | 1.1% |
| 39.761° | 2.26518 Å | 0.2% |

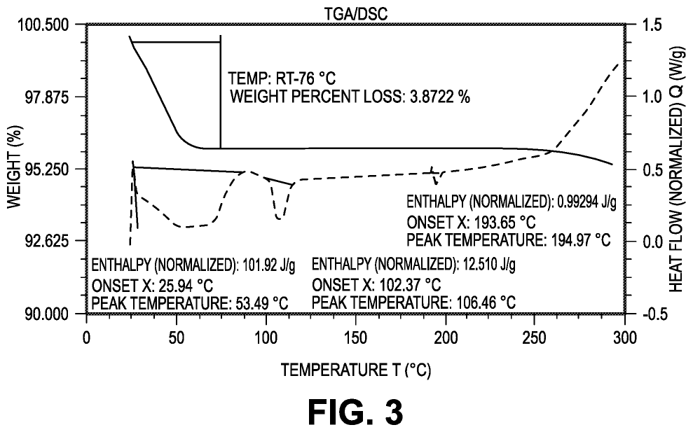

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 3. In some embodiments, the crystalline form exhibits less than about 4.9%±0.5 weight loss up to 76° C.±10.0 as determined by thermogravimetric analysis. In some embodiments, the crystalline form exhibits less than about 3.9%±0.5 weight loss up to 76° C.±10.0 as determined by thermogravimetric analysis. In some embodiments, the crystalline form exhibits at least about 2.9%±0.5 weight loss up to 76° C.±10.0 as determined by thermogravimetric analysis.

Provided herein is crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by endothermic peaks at 53.5° C.±10.0, 106.5° C.±5.0, and 193.7° C.±5.0 as shown in FIG. 3. In some embodiments, the DSC is characterized by an endothermic peak 106.5° C.±5.0.

In some embodiments, provided herein is a composition wherein the crystalline Form I is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form I is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form II is characterized by exhibits an X-ray powder diffraction reflection at a 2-theta value of 11.8°±0.3. In some embodiments, the crystalline form exhibits an X-ray powder diffraction reflection at a 2-theta value of 7.9°±0.3 and 13.7°±0.3. In some embodiments, the crystalline form exhibits an X-ray powder diffraction reflection at a 2-theta value of 9.5°±0.3, 18.9°±0.3, and 26.8°±0.3. In some embodiments, the crystalline form exhibits an X-ray powder diffraction reflection at a 2-theta value of 15.9°±0.3, 20.5°±0.3, and 22.3°±0.3. In some embodiments, the crystalline form exhibits an X-ray powder diffraction reflection at a 2-theta value of 20.8°±0.3, 25.7°±0.3, and 26.3°±0.3.

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form I is characterized by at least one X-ray diffraction pattern reflection selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the crystalline Form I is characterized by at least two X-ray diffraction pattern reflections selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the crystalline Form II is characterized by at least three X-ray diffraction pattern reflections selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the crystalline Form II is characterized by at least four X-ray diffraction pattern reflections selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the crystalline Form II is characterized by at least five X-ray diffraction pattern reflections selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3. In some embodiments, the crystalline Form II is characterized by at least six X-ray diffraction pattern reflections selected from a 2 theta value of 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

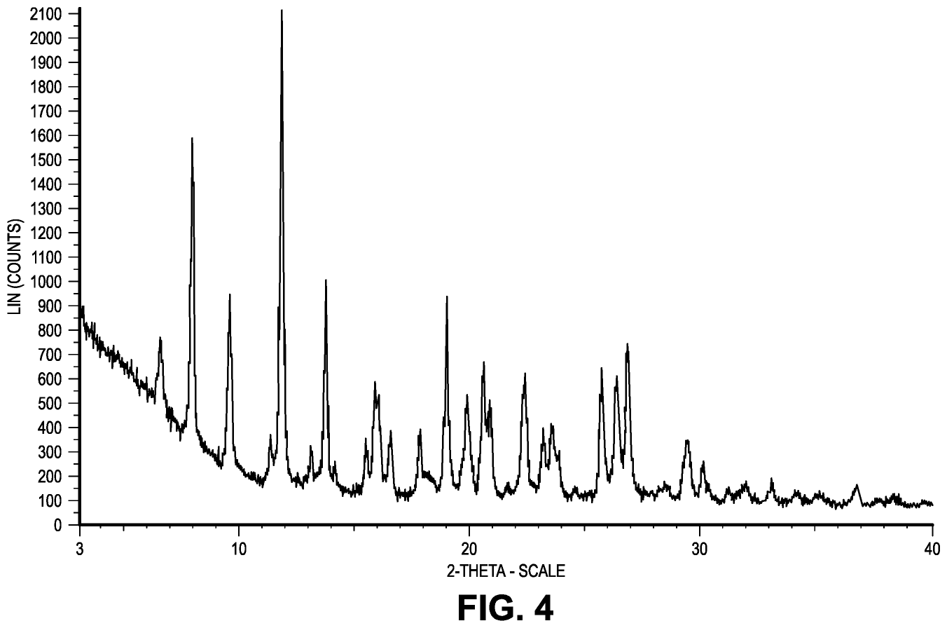

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 4.

TABLE 2
Peak listing for the X-ray powder diffractogram
of the crystalline solid state form of Form II.

| | | Rel. |
|---|---|---|
| Angle | d Value | Intensity |
| 6.465° | 13.66052 Å | 12.0% |
| 7.852° | 11.25009 Å | 62.0% |
| 9.484° | 9.31796 Å | 36.5% |
| 11.272° | 7.84321 Å | 10.2% |
| 11.750° | 7.52549 Å | 100.0% |
| 13.024° | 6.79233 Å | 7.8% |
| 13.664° | 6.47532 Å | 44.3% |
| 14.050° | 6.29832 Å | 4.6% |
| 15.419° | 5.74191 Å | 11.1% |
| 15.864° | 5.58185 Å | 16.6% |
| 16.491° | 5.37118 Å | 13.9% |
| 17.771° | 4.98694 Å | 13.0% |
| 18.929° | 4.68442 Å | 43.5% |
| 19.803° | 4.47975 Å | 19.6% |
| 20.541° | 4.32026 Å | 26.9% |
| 20.797° | 4.26785 Å | 20.8% |
| 21.565° | 4.11742 Å | 2.1% |
| 22.327° | 3.97857 Å | 26.7% |
| 23.128° | 3.84263 Å | 15.1% |
| 23.508° | 3.78130 Å | 14.4% |
| 23.764° | 3.74115 Å | 7.8% |
| 24.514° | 3.62836 Å | 2.0% |
| 25.681° | 3.46615 Å | 26.9% |
| 26.315° | 3.38397 Å | 25.7% |
| 26.785° | 3.32566 Å | 33.1% |
| 29.403° | 3.03523 Å | 11.2% |
| 30.062° | 2.97021 Å | 7.5% |
| 31.189° | 2.86544 Å | 3.2% |
| 31.946° | 2.79921 Å | 5.0% |
| 33.078° | 2.70599 Å | 5.9% |
| 34.144° | 2.62389 Å | 1.7% |
| 36.746° | 2.44383 Å | 4.5% |

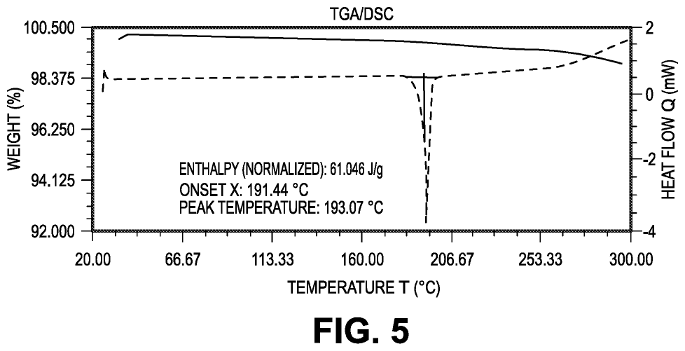

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 5. In some embodiments, the crystalline form exhibits less than 0.1%±0.5 weight loss up to 200° C.±10.0 as determined by thermogravimetric analysis. In some embodiments, the crystalline form exhibits less than 1.1%±0.5 weight loss up to 200° C.±10.0 as determined by thermogravimetric analysis.

Provided herein is crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by a an endothermic peak at 193.1° C.±5.0 as shown in FIG. 5.

In some embodiments, provided herein is a composition wherein the crystalline Form II is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form II is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form III is characterized by exhibits an X-ray powder diffraction reflection at a 2-theta value of 8.7°±0.3. In some embodiments, the crystalline form exhibits an X-ray powder diffraction reflection at a 2-theta value of 5.0°±0.3 and 12.8°±0.3. In some embodiments, the crystalline form exhibits an X-ray powder diffraction reflection at a 2-theta value of 23.9°±0.3 and 24.3°±0.3. In some embodiments, the crystalline form exhibits an X-ray powder diffraction reflection at a 2-theta value of 11.5°±0.3, 20.3°±0.3, and 21.2°±0.3. In some embodiments, the crystalline form exhibits an X-ray powder diffraction reflection at a 2-theta value of 11.6°±0.3, 15.9°±0.3, and 25.8°±0.3.

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form III is characterized by at least one X-ray diffraction pattern reflection selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the crystalline Form III is characterized by at least two X-ray diffraction pattern reflections selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the crystalline Form III is characterized by at least three X-ray diffraction pattern reflections selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the crystalline Form III is characterized by at least four X-ray diffraction pattern reflections selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the crystalline Form III is characterized by at least five X-ray diffraction pattern reflections selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3. In some embodiments, the crystalline Form III is characterized by at least six X-ray diffraction pattern reflections selected from a 2 theta value of 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

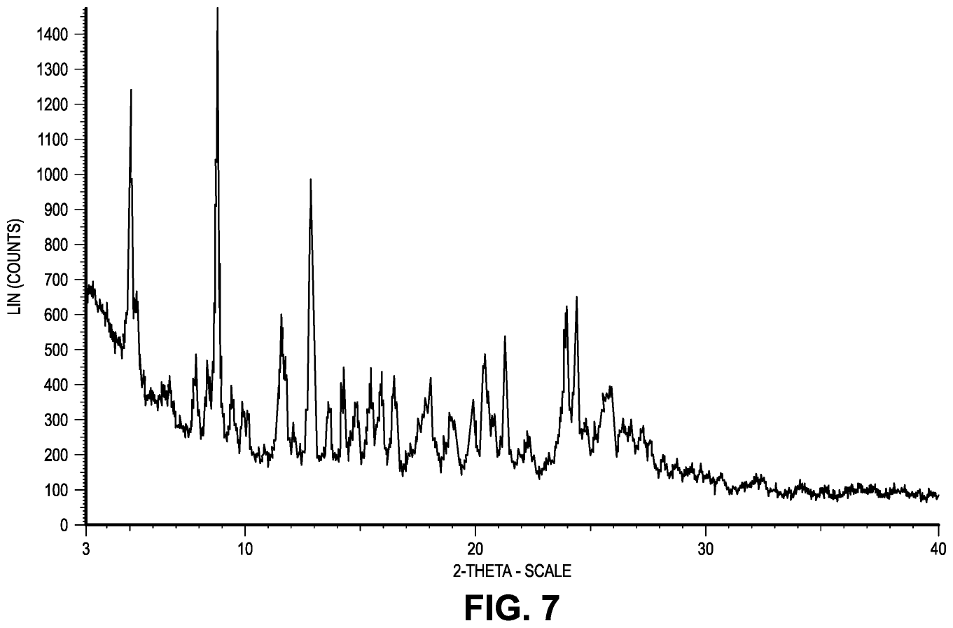

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 7.

TABLE 3
Peak listing for the X-ray powder diffractogram
of the crystalline solid state form of Form III.

| | d | Rel. |
|---|---|---|
| Angle | Value | Intensity |
| 4.947° | 17.84963 Å | 56.3% |
| 5.148° | 17.15250 Å | 15.6% |
| 6.610° | 13.36200 Å | 9.1% |
| 7.753° | 11.39423 Å | 19.3% |
| 8.281° | 10.66901 Å | 15.5% |
| 8.692° | 10.16555 Å | 100.0% |
| 9.348° | 9.45269 Å | 9.9% |
| 9.812° | 9.00680 Å | 9.7% |
| 10.030° | 8.81180 Å | 7.2% |
| 11.474° | 7.70623 Å | 31.0% |
| 11.632° | 7.60126 Å | 22.8% |
| 12.010° | 7.36316 Å | 6.2% |
| 12.755° | 6.93471 Å | 64.2% |
| 13.537° | 6.53600 Å | 11.7% |
| 14.171° | 6.24466 Å | 20.3% |
| 14.709° | 6.01745 Å | 12.0% |
| 15.374° | 5.75889 Å | 21.6% |
| 15.843° | 5.58918 Å | 23.0% |
| 16.373° | 5.40942 Å | 19.9% |
| 17.450° | 5.07817 Å | 9.7% |
| 17.945° | 4.93913 Å | 21.9% |
| 18.542° | 4.78127 Å | 8.3% |
| 18.858° | 4.70186 Å | 10.9% |
| 18.835° | 4.70770 Å | 9.8% |
| 19.801° | 4.48011 Å | 17.0% |
| 20.320° | 4.36675 Å | 24.7% |
| 20.490° | 4.33109 Å | 10.9% |
| 20.684° | 4.29079 Å | 8.8% |
| 21.221° | 4.18346 Å | 33.1% |
| 22.205° | 4.00019 Å | 5.5% |
| 23.865° | 3.72561 Å | 32.4% |
| 24.306° | 3.65892 Å | 40.7% |
| 24.711° | 3.59998 Å | 9.1% |
| 25.143° | 3.53900 Å | 4.9% |
| 25.728° | 3.45990 Å | 18.1% |
| 26.309° | 3.38482 Å | 9.0% |
| 26.348° | 3.37980 Å | 7.0% |
| 26.686° | 3.33784 Å | 11.7% |
| 27.166° | 3.27988 Å | 9.0% |
| 27.516° | 3.23895 Å | 6.1% |
| 28.112° | 3.17164 Å | 3.9% |
| 28.658° | 3.11244 Å | 4.4% |
| 29.717° | 3.00395 Å | 3.5% |
| 30.595° | 2.91972 Å | 4.1% |

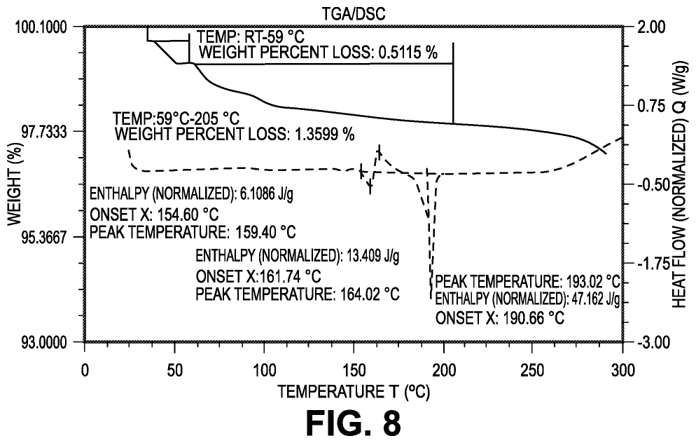

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 8. In some embodiments, the crystalline form exhibits less than 2.9%±0.5 weight loss up to 205° C.±10.0 as determined by thermogravimetric analysis. In some embodiments, the crystalline form exhibits less than 1.9%±0.5 weight loss up to 205° C.±10.0 as determined by thermogravimetric analysis. In some embodiments, the crystalline form exhibits at least 0.9%±0.5 weight loss up to 205° C.±10.0 as determined by thermogravimetric analysis.

Provided herein is crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized endothermic peaks at 164.0° C.±5.0 and 190.6° C.±5.0 as shown in FIG. 8. In some embodiments, the DSC is characterized by an endothermic peak at 190.6° C.±5.0

In some embodiments, provided herein is a composition wherein the crystalline Form III is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form III is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form IV is characterized by exhibits an X-ray powder diffraction reflection at a 2-theta value of 5.1°±0.3. In some embodiments, the crystalline form exhibits an X-ray powder diffraction reflection at a 2-theta value of 10.2°±0.3 and 8.6°±0.3. In some embodiments, the crystalline form exhibits an X-ray powder diffraction reflection at a 2-theta value of 12.2°±0.3 and 17.3°±0.3. In some embodiments, the crystalline form exhibits an X-ray powder diffraction reflection at a 2-theta value of 6.4°±0.3, 13.4°±0.3, and 15.3°±0.3. In some embodiments, the crystalline form exhibits an X-ray powder diffraction reflection at a 2-theta value of 15.8°±0.3, and 26.0°±0.3.

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form IV is characterized by at least one X-ray diffraction pattern reflection selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the crystalline Form IV is characterized by at least two X-ray diffraction pattern reflections selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the crystalline Form IV is characterized by at least three X-ray diffraction pattern reflections selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the crystalline Form IV is characterized by at least four X-ray diffraction pattern reflections selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the crystalline Form IV is characterized by at least five X-ray diffraction pattern reflections selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3. In some embodiments, the crystalline Form IV is characterized by at least six X-ray diffraction pattern reflections selected from a 2 theta value of 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

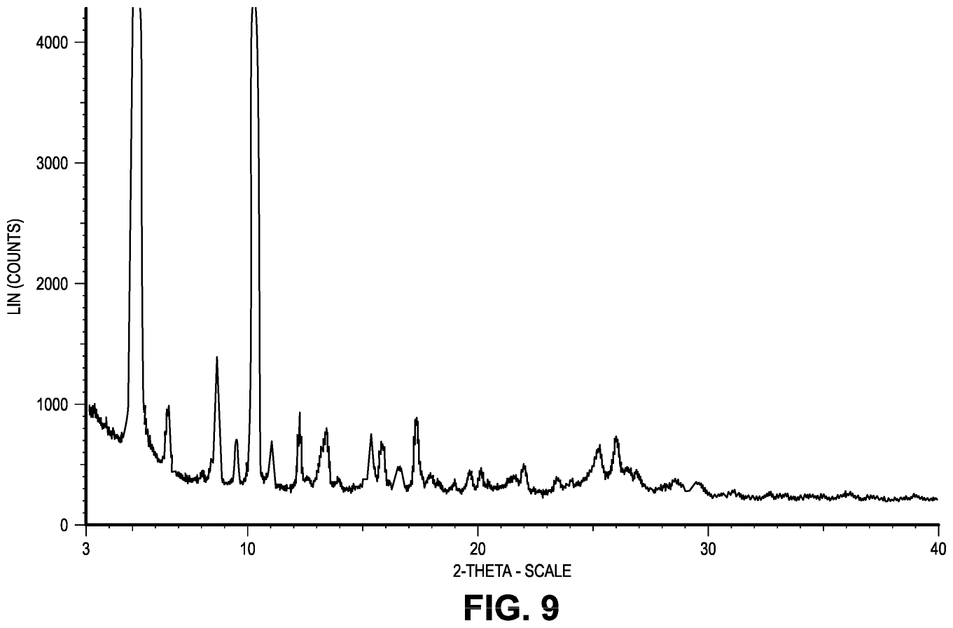

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 9.

TABLE 4
Peak listing for the X-ray powder diffractogram
of the crystalline solid state form of Form IV.

| | d | Rel. |
|---|---|---|
| Angle | Value | Intensity |
| 5.063° | 17.43962 Å | 100.0% |
| 6.434° | 13.72583 Å | 2.5% |
| 7.935° | 11.13263 Å | 0.4% |
| 8.580° | 10.29804 Å | 5.4% |
| 9.413° | 9.38766 Å | 1.8% |
| 10.189° | 8.67505 Å | 25.1% |
| 10.938° | 8.08234 Å | 2.0% |
| 12.174° | 7.26460 Å | 3.3% |
| 12.520° | 7.06440 Å | 0.4% |
| 13.128° | 6.73853 Å | 1.8% |
| 13.355° | 6.62459 Å | 2.6% |
| 13.841° | 6.39315 Å | 0.5% |
| 15.315° | 5.78084 Å | 2.4% |
| 15.739° | 5.62617 Å | 2.0% |
| 16.491° | 5.37097 Å | 0.9% |
| 17.265° | 5.13216 Å | 3.2% |
| 17.887° | 4.95502 Å | 0.7% |
| 18.957° | 4.67754 Å | 0.6% |
| 19.608° | 4.52371 Å | 0.8% |
| 20.085° | 4.41743 Å | 0.8% |
| 21.312° | 4.16570 Å | 0.4% |
| 21.551° | 4.12009 Å | 0.5% |
| 21.963° | 4.04376 Å | 1.1% |
| 23.413° | 3.79652 Å | 0.5% |
| 25.265° | 3.52229 Å | 1.7% |
| 25.980° | 3.42683 Å | 2.0% |
| 26.474° | 3.36405 Å | 0.6% |
| 26.876° | 3.31460 Å | 0.8% |
| 28.559° | 3.12298 Å | 0.4% |
| 29.503° | 3.02516 Å | 0.3% |
| 29.605° | 3.01500 Å | 0.4% |
| 31.105° | 2.87296 Å | 0.2% |
| 32.706° | 2.73588 Å | 0.3% |

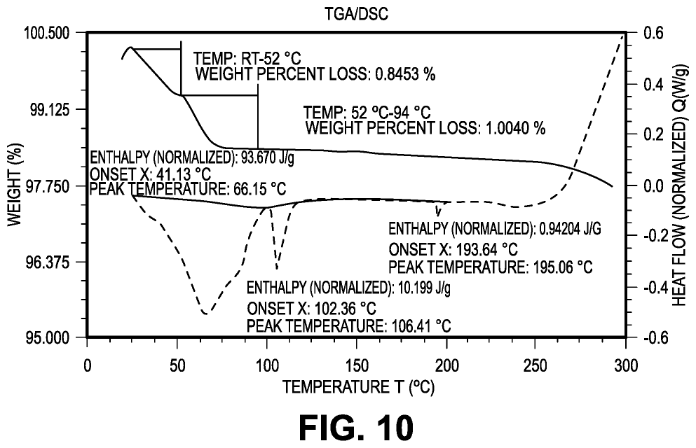

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 10. In some embodiments, the crystalline form exhibits less than 2.9%±0.5 weight loss up to 94° C.±10.0 as determined by thermogravimetric analysis. In some embodiments, the crystalline form exhibits less than 1.9%±0.5 weight loss up to 94° C.±10.0 as determined by thermogravimetric analysis. In some embodiments, the crystalline form exhibits at least 0.9%±0.5 weight loss up to 94° C.±10.0 as determined by thermogravimetric analysis.

Provided herein is crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by endothermic peaks at 66.2° C.±5.0, 106.4° C.±5.0, and 193.6° C.±5.0 as shown in FIG. 10. In some embodiments, the DSC is characterized by a endothermic peak at 106.4° C.±5.0.

In some embodiments, provided herein is a composition wherein the crystalline Form IV is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form IV is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form Vis characterized by exhibits an X-ray powder diffraction reflection at a 2-theta value of 5.1°±0.3. In some embodiments, the crystalline form exhibits an X-ray powder diffraction reflection at a 2-theta value of 10.4°±0.3. In some embodiments, the crystalline form exhibits an X-ray powder diffraction reflection at a 2-theta value of 6.4°±0.3 and 25.6°±0.3. In some embodiments, the crystalline form exhibits an X-ray powder diffraction reflection at a 2-theta value of 8.7°±0.3, 11.9°±0.3, and 15.6°±0.3. In some embodiments, the crystalline form exhibits an X-ray powder diffraction reflection at a 2-theta value of 13.5°±0.3, and 25.8°±0.3.

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form Vis characterized by at least one X-ray diffraction pattern reflection selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the crystalline Form V is characterized by at least two X-ray diffraction pattern reflections selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the crystalline Form V is characterized by at least three X-ray diffraction pattern reflections selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the crystalline Form Vis characterized by at least four X-ray diffraction pattern reflections selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the crystalline Form V is characterized by at least five X-ray diffraction pattern reflections selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3. In some embodiments, the crystalline Form V is characterized by at least six X-ray diffraction pattern reflections selected from a 2 theta value of 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

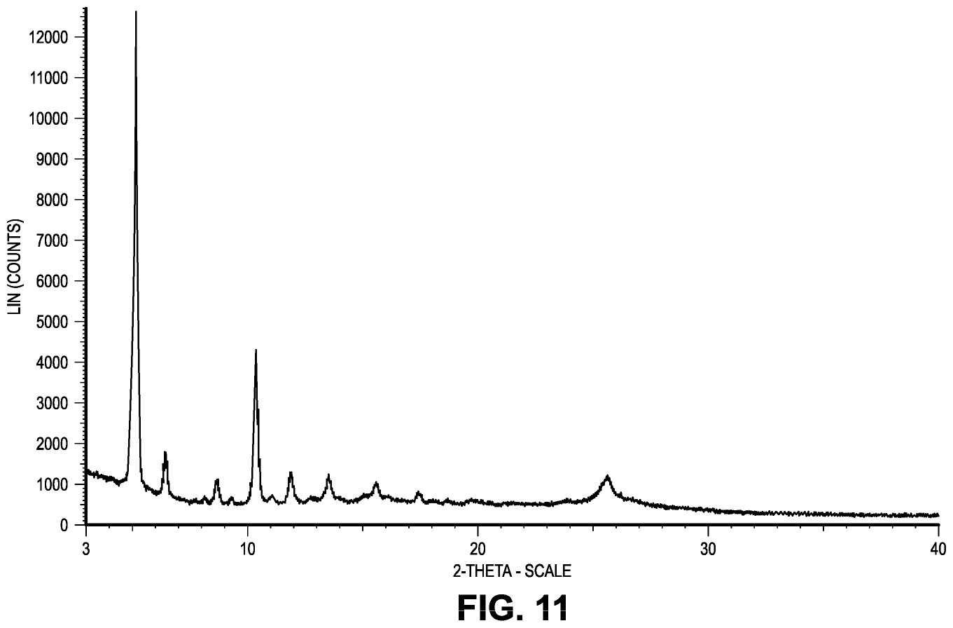

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 11.

TABLE 5
Peak listing for the X-ray powder diffractogram
of the crystalline solid state form of Form V.

| | d | Rel. |
|---|---|---|
| Angle | Value | Intensity |
| 5.130° | 17.21082 Å | 100.0% |
| 6.422° | 13.75299 Å | 8.9% |
| 8.116° | 10.88471 Å | 1.4% |
| 8.659° | 10.20331 Å | 5.2% |
| 9.287° | 9.51492 Å | 1.6% |
| 10.362° | 8.52992 Å | 34.8% |
| 11.049° | 8.00161 Å | 1.9% |
| 11.878° | 7.44488 Å | 6.9% |
| 12.854° | 6.88132 Å | 0.9% |
| 12.892° | 6.86141 Å | 1.2% |
| 13.528° | 6.54015 Å | 6.1% |
| 15.118° | 5.85576 Å | 0.7% |
| 15.582° | 5.68227 Å | 4.3% |
| 16.045° | 5.51928 Å | 0.7% |
| 17.433° | 5.08304 Å | 2.8% |
| 18.737° | 4.73211 Å | 1.5% |
| 25.773° | 3.45392 Å | 4.4% |
| 25.642° | 3.47125 Å | 6.4% |

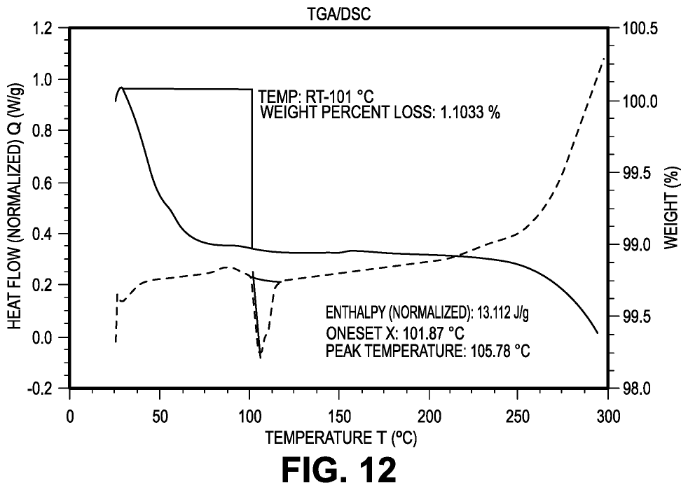

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 12. In some embodiments, the crystalline form exhibits less than 2.1%±0.5 weight loss up to 101° C.±10.0 as determined by thermogravimetric analysis. In some embodiments, the crystalline form exhibits less than 1.1%±0.5 weight loss up to 101° C.±10.0 as determined by thermogravimetric analysis. In some embodiments, the crystalline form exhibits at least 0.6%±0.5 weight loss up to 101° C.±10.0 as determined by thermogravimetric analysis.

Provided herein is crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by an endothermic peak at 105.8° C.±5.0 as shown in FIG. 12.

In some embodiments, provided herein is a composition wherein the crystalline Form V is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form V is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VI is characterized by exhibits an X-ray powder diffraction reflection at a 2-theta value of 9.5°±0.3. In some embodiments, the crystalline form exhibits an X-ray powder diffraction reflection at a 2-theta value of 5.2°±0.3 and 23.7°±0.3. In some embodiments, the crystalline form exhibits an X-ray powder diffraction reflection at a 2-theta value of 5.7°±0.3, 10.2°±0.3, and 19.5°±0.3. In some embodiments, the crystalline form exhibits an X-ray powder diffraction reflection at a 2-theta value of 6.5°±0.3, 15.5°±0.3, and 26.8°±0.3. In some embodiments, the crystalline form exhibits an X-ray powder diffraction reflection at a 2-theta value of 7.4°±0.3, 15.0°±0.3, 19.0°±0.3, and 24.7°±0.3.

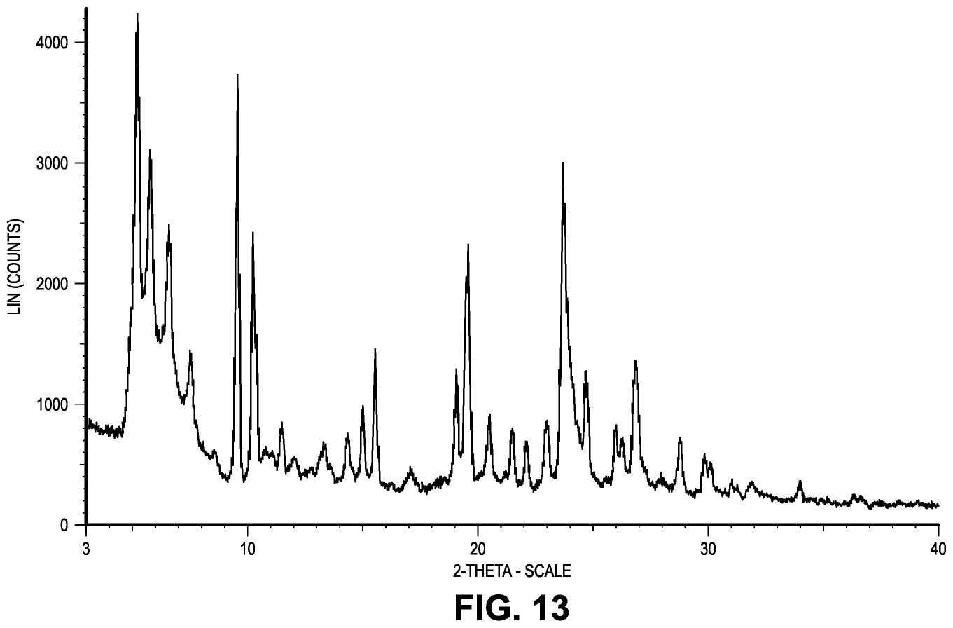

Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VI is characterized by at least one X-ray diffraction pattern reflection selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the crystalline Form VI is characterized by at least two X-ray diffraction pattern reflections selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the crystalline Form VI is characterized by at least three X-ray diffraction pattern reflections selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the crystalline Form VI is characterized by at least four X-ray diffraction pattern reflections selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the crystalline Form VI is characterized by at least five X-ray diffraction pattern reflections selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. In some embodiments, the crystalline Form VI is characterized by at least six X-ray diffraction pattern reflections selected from a 2 theta value of 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3. Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 13.

TABLE 6
Peak listing for the X-ray powder diffractogram
of the crystalline solid state form of Form VI.

| | d | Rel. |
|---|---|---|
| Angle | Value | Intensity |
| 5.118° | 17.25220 Å | 96.8% |
| 5.661° | 15.59764 Å | 68.1% |
| 6.480° | 13.62986 Å | 51.7% |
| 7.426° | 11.89546 Å | 25.9% |
| 8.449° | 10.45656 Å | 4.7% |
| 9.473° | 9.32850 Å | 100.0% |
| 10.160° | 8.69938 Å | 59.7% |
| 10.694° | 8.26621 Å | 7.1% |
| 10.936° | 8.08352 Å | 5.4% |
| 11.407° | 7.75083 Å | 13.5% |
| 11.966° | 7.39015 Å | 4.7% |
| 12.710° | 6.95911 Å | 2.5% |
| 13.249° | 6.67733 Å | 9.6% |
| 14.276° | 6.19910 Å | 12.0% |
| 14.949° | 5.92168 Å | 18.6% |
| 15.481° | 5.71904 Å | 33.3% |
| 17.018° | 5.20585 Å | 5.8% |
| 19.015° | 4.66351 Å | 29.9% |
| 19.528° | 4.54219 Å | 60.5% |
| 20.454° | 4.33846 Å | 17.0% |
| 21.145° | 4.19832 Å | 3.7% |
| 21.464° | 4.13670 Å | 14.8% |
| 22.078° | 4.02288 Å | 10.9% |
| 22.959° | 3.87056 Å | 15.1% |
| 23.673° | 3.75533 Å | 81.8% |
| 24.355° | 3.65178 Å | 12.7% |
| 24.678° | 3.60461 Å | 29.6% |
| 25.978° | 3.42711 Å | 14.7% |
| 26.218° | 3.39632 Å | 12.4% |
| 26.821° | 3.32138 Å | 31.3% |
| 27.309° | 3.26304 Å | 3.4% |
| 27.944° | 3.19029 Å | 3.3% |
| 28.794° | 3.09806 Å | 12.3% |
| 29.839° | 2.99193 Å | 10.4% |
| 30.119° | 2.96477 Å | 7.5% |
| 31.038° | 2.87898 Å | 4.0% |
| 31.284° | 2.85696 Å | 3.4% |
| 31.919° | 2.80150 Å | 4.2% |
| 34.018° | 2.63328 Å | 5.3% |
| 36.369° | 2.46830 Å | 2.9% |
| 36.671° | 2.44865 Å | 2.6% |

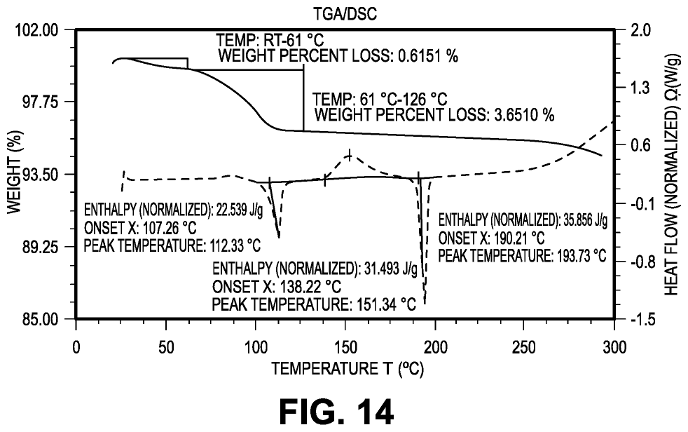

Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 14. In some embodiments, the crystalline form exhibits less than 5.3%±0.5 weight loss up to 126° C.±10.0 as determined by thermogravimetric analysis. In some embodiments, the crystalline form exhibits less than 4.3%±0.5 weight loss up to 126° C.±10.0 as determined by thermogravimetric analysis. In some embodiments, the crystalline form exhibits at least 3.3%±0.5 weight loss up to 126° C.±10.0 as determined by thermogravimetric analysis.

Provided herein is crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by endothermic peaks at 112.3° C.±5.0 and 193.7° C.±5.0 as shown in FIG. 14. In some embodiments, the DSC is characterized by a endothermic peak at 193.7° C.±5.0. In some embodiments, the DSC is characterized by a exothermic peak at 151° C.±5.0

In some embodiments, provided herein is a composition wherein the crystalline Form VI is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form VI is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VII is characterized by exhibits an X-ray powder diffraction reflection at a 2-theta value of 5.4°±0.3. In some embodiments, the crystalline form exhibits an X-ray powder diffraction reflection at a 2-theta value of 9.8°±0.3 and 23.4°±0.3. In some embodiments, the crystalline form exhibits an X-ray powder diffraction reflection at a 2-theta value of 5.0°±0.3 and 15.2°±0.3. In some embodiments, the crystalline form exhibits an X-ray powder diffraction reflection at a 2-theta value of 10.1°±0.3, 13.6°±0.3, and 26.7°±0.3. In some embodiments, the crystalline form exhibits an X-ray powder diffraction reflection at a 2-theta value of 9.1°±0.3, 25.6°±0.3, and 27.2°±0.3.

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VII is characterized by at least one X-ray diffraction pattern reflection selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the crystalline Form VII is characterized by at least two X-ray diffraction pattern reflections selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the crystalline Form VII is characterized by at least three X-ray diffraction pattern reflections selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the crystalline Form VII is characterized by at least four X-ray diffraction pattern reflections selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the crystalline Form VII is characterized by at least five X-ray diffraction pattern reflections selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3. In some embodiments, the crystalline Form VII is characterized by at least six X-ray diffraction pattern reflections selected from a 2 theta value of 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

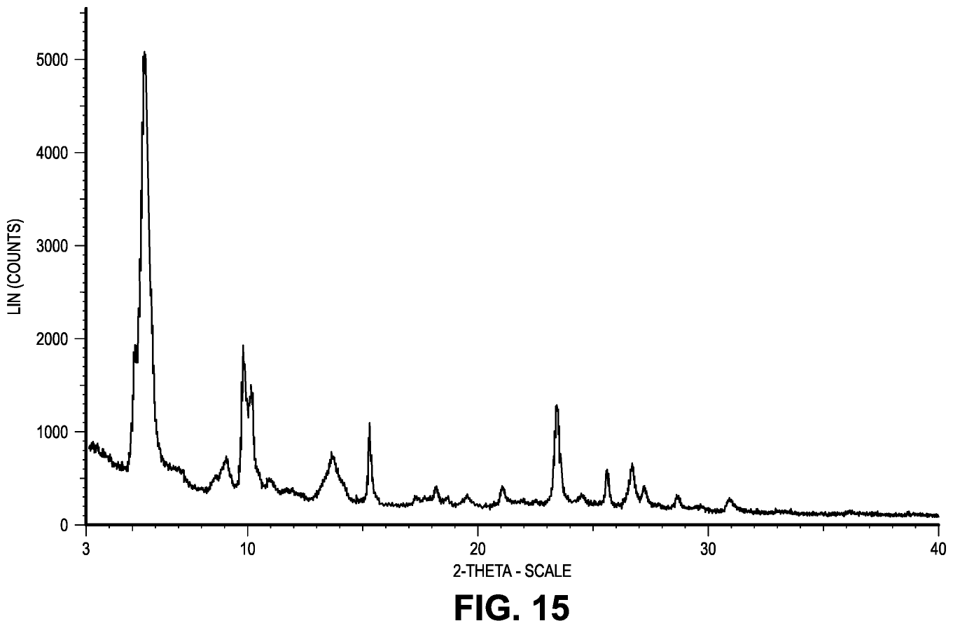

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 15.

TABLE 7
Peak listing for the X-ray powder diffractogram
of the crystalline solid state form of Form VII.

| | d | Rel. |
|---|---|---|
| Angle | Value | Intensity |
| 5.011° | 17.62041 Å | 27.4% |
| 5.411° | 16.32039 Å | 100.0% |
| 6.951° | 12.70685 Å | 2.4% |
| 8.517° | 10.37300 Å | 4.2% |
| 9.009° | 9.80757 Å | 6.2% |
| 9.770° | 9.04571 Å | 30.4% |
| 10.084° | 8.76462 Å | 25.2% |
| 10.887° | 8.12038 Å | 3.4% |
| 13.598° | 6.50642 Å | 12.0% |
| 15.233° | 5.81186 Å | 20.6% |
| 18.127° | 4.89002 Å | 4.2% |
| 18.638° | 4.75703 Å | 2.3% |
| 19.521° | 4.54372 Å | 2.8% |
| 21.029° | 4.22119 Å | 4.8% |
| 23.404° | 3.79791 Å | 26.8% |
| 24.482° | 3.63307 Å | 1.6% |
| 25.589° | 3.47833 Å | 9.6% |
| 26.693° | 3.33700 Å | 11.2% |
| 27.204° | 3.27541 Å | 5.3% |
| 28.644° | 3.11397 Å | 2.9% |
| 30.906° | 2.89104 Å | 3.6% |
| 36.172° | 2.48132 Å | 1.2% |

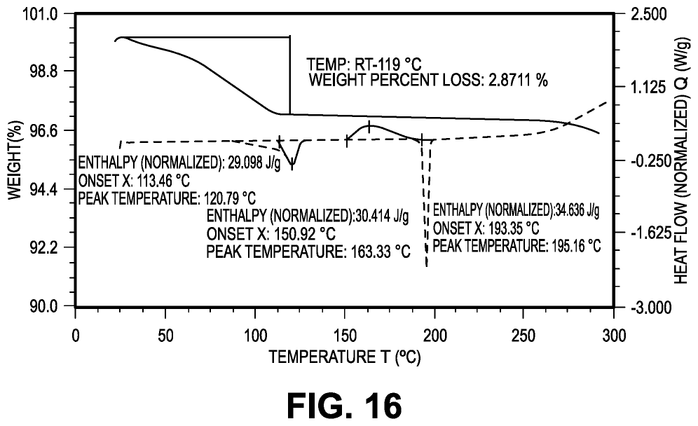

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 16. In some embodiments, the crystalline form exhibits less than 3.9%±0.5 weight loss up to 119° C. #10.0 as determined by thermogravimetric analysis. In some embodiments, the crystalline form exhibits less than 2.9%±0.5 weight loss up to 119° C.±10.0 as determined by thermogravimetric analysis. In some embodiments, the crystalline form exhibits at least 1.9%±0.5 weight loss up to 119° C.±10.0 as determined by thermogravimetric analysis.

Provided herein is crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by endothermic peaks at 120.8° C.±5.0 and 195.1° C.±5.0 as shown in FIG. 16. In some embodiments, the DSC is characterized by an endothermic peak 195.1° C.±5.0 In some embodiments, the DSC is characterized by an exothermic peak 163.3° C. #5.0.

In some embodiments, provided herein is a composition wherein the crystalline Form VII is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form VII is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VIII is characterized by exhibits an X-ray powder diffraction reflection at a 2-theta value of 5.8°±0.3. In some embodiments, the crystalline form exhibits an X-ray powder diffraction reflection at a 2-theta value of 5.2°±0.3 and 15.2°±0.3. In some embodiments, the crystalline form exhibits an X-ray powder diffraction reflection at a 2-theta value of 5.0°±0.3 and 10.1°±0.3. In some embodiments, the crystalline form exhibits an X-ray powder diffraction reflection at a 2-theta value of 8.4°±0.3, 9.8°±0.3, and 23.4°±0.3. In some embodiments, the crystalline form exhibits an X-ray powder diffraction reflection at a 2-theta value of 10.4°±0.3, 14.2°±0.3, and 25.5°±0.3.

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form VIII is characterized by at least one X-ray diffraction pattern reflection selected from a 2 theta value of 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the crystalline Form VIII is characterized by at least two X-ray diffraction pattern reflections selected from a 2 theta value of 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the crystalline Form VIII is characterized by at least three X-ray diffraction pattern reflections selected from a 2 theta value of 5.0°±0.3, 5.2°±0.3, 5.8°±=0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the crystalline Form VIII is characterized by at least four X-ray diffraction pattern reflections selected from a 2 theta value of 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the crystalline Form VIII is characterized by at least five X-ray diffraction pattern reflections selected from a 2 theta value of 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3. In some embodiments, the crystalline Form VIII is characterized by at least six X-ray diffraction pattern reflections selected from a 2 theta value of 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

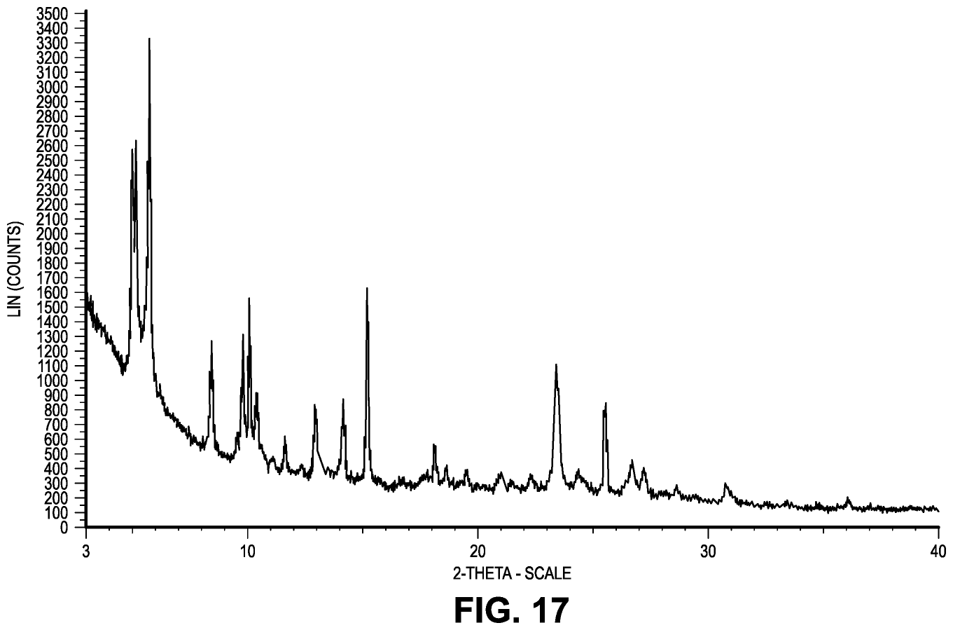

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 17.

TABLE 8
Peak listing for the X-ray powder diffractogram
of the crystalline solid state form of Form VIII.

| | d | Rel. |
|---|---|---|
| Angle | Value | Intensity |
| 4.980° | 17.72969 Å | 57.8% |
| 5.154° | 17.13212 Å | 64.4% |
| 5.746° | 15.36778 Å | 100.0% |
| 8.418° | 10.49541 Å | 31.3% |
| 9.541° | 9.26238 Å | 6.7% |
| 9.790° | 9.02768 Å | 38.3% |
| 10.076° | 8.77188 Å | 48.7% |
| 10.392° | 8.50586 Å | 22.2% |
| 11.081° | 7.97852 Å | 3.6% |
| 11.620° | 7.60953 Å | 10.0% |
| 12.306° | 7.18670 Å | 3.0% |
| 12.933° | 6.83954 Å | 21.1% |
| 14.167° | 6.24659 Å | 25.9% |
| 15.192° | 5.82717 Å | 60.9% |
| 18.143° | 4.88550 Å | 11.9% |
| 18.629° | 4.75914 Å | 6.5% |
| 19.490° | 4.55090 Å | 6.1% |
| 20.989° | 4.22923 Å | 4.1% |
| 21.476° | 4.13425 Å | 2.8% |
| 22.311° | 3.98144 Å | 3.4% |
| 23.417° | 3.79584 Å | 37.5% |
| 24.364° | 3.65040 Å | 7.4% |
| 25.535° | 3.48561 Å | 25.6% |
| 26.685° | 3.33795 Å | 10.4% |
| 27.198° | 3.27612 Å | 8.4% |
| 28.648° | 3.11350 Å | 3.3% |
| 30.780° | 2.90253 Å | 7.3% |
| 36.087° | 2.48693 Å | 1.8% |

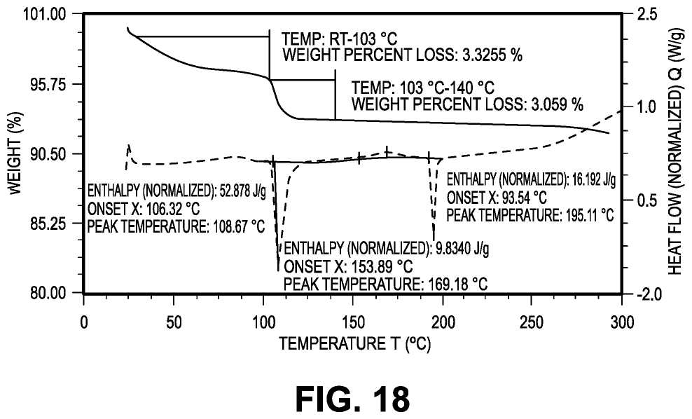

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 18. In some embodiments, the crystalline form exhibits less than 7.4%±0.5 weight loss up to 140° C.±10.0 as determined by thermogravimetric analysis. In some embodiments, the crystalline form exhibits less than 6.4%±0.5 weight loss up to 140° C.±10.0 as determined by thermogravimetric analysis. In some embodiments, the crystalline form exhibits at least 5.4%±0.5 weight loss up to 140° C.±10.0 as determined by thermogravimetric analysis.

Provided herein is crystalline Form VIII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by endothermic peaks at 108.7° C.±5.0 and 195.1° C.±5.0 as shown in FIG. 18. In some embodiments, the DSC is characterized by endothermic peaks at 108.7° C.±5.0 or 195.1° C.±5.0. In some embodiments, the DSC is characterized by an exothermic peak at 169.2.

In some embodiments, provided herein is a composition wherein the crystalline Form VIII is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form VIII is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide.

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form IX is characterized by exhibits an X-ray powder diffraction reflection at a 2-theta value of 12.3°±0.3. In some embodiments, the crystalline form exhibits an X-ray powder diffraction reflection at a 2-theta value of 6.1°±0.3 and 13.4°±0.3. In some embodiments, the crystalline form exhibits an X-ray powder diffraction reflection at a 2-theta value of 7.9°±0.3 and 10.5°±0.3. In some embodiments, the crystalline form exhibits an X-ray powder diffraction reflection at a 2-theta value of 11.8°±0.3, 25.0°±0.3, and 25.7°±0.3. In some embodiments, the crystalline form exhibits an X-ray powder diffraction reflection at a 2-theta value of 18.6°±0.3, 23.0°±0.3, and 25.2°±0.3.

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the crystalline Form IX is characterized by at least one X-ray diffraction pattern reflection selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the crystalline Form IX is characterized by at least two X-ray diffraction pattern reflections selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the crystalline Form IX is characterized by at least three X-ray diffraction pattern reflections selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the crystalline Form IX is characterized by at least four X-ray diffraction pattern reflections selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the crystalline Form IX is characterized by at least five X-ray diffraction pattern reflections selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3. In some embodiments, the crystalline Form IX is characterized by at least six X-ray diffraction pattern reflections selected from a 2 theta value of 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

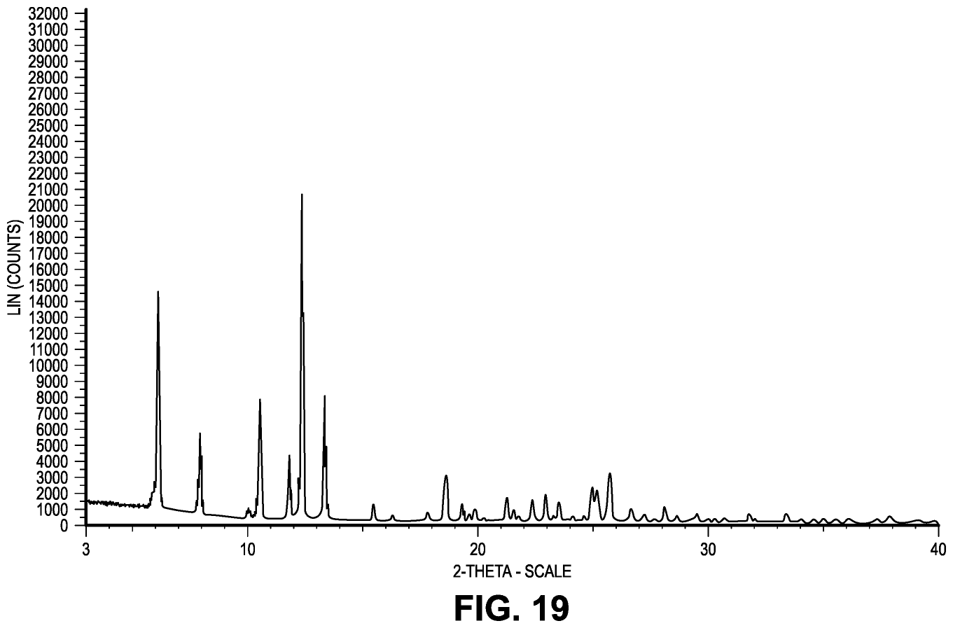

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the X-ray powder diffraction pattern as shown in FIG. 19.

TABLE 9
Peak listing for the X-ray powder diffractogram
of the crystalline solid state form of Form IX.

| | d | Rel. |
|---|---|---|
| Angle | Value | Intensity |
| 5.875° | 15.03174 Å | 4.7% |
| 6.101° | 14.47472 Å | 62.2% |
| 7.916° | 11.16015 Å | 23.9% |
| 10.031° | 8.81127 Å | 2.8% |
| 10.517° | 8.40485 Å | 35.6% |
| 11.801° | 7.49288 Å | 19.3% |
| 12.345° | 7.16409 Å | 100.0% |
| 13.352° | 6.62584 Å | 37.5% |
| 13.661° | 6.47673 Å | 0.3% |
| 15.448° | 5.73118 Å | 4.8% |
| 16.298° | 5.43422 Å | 1.4% |
| 17.798° | 4.97964 Å | 2.5% |
| 18.616° | 4.76243 Å | 14.2% |
| 19.323° | 4.58990 Å | 5.3% |
| 19.621° | 4.52084 Å | 1.6% |
| 19.867° | 4.46542 Å | 3.4% |
| 20.275° | 4.37637 Å | 0.6% |
| 20.506° | 4.32765 Å | 0.3% |
| 20.949° | 4.23722 Å | 0.5% |
| 21.248° | 4.17815 Å | 7.5% |
| 21.542° | 4.12189 Å | 3.5% |
| 21.762° | 4.08071 Å | 1.2% |
| 22.362° | 3.97255 Å | 7.0% |
| 22.949° | 3.87216 Å | 8.8% |
| 23.328° | 3.81015 Å | 0.9% |
| 23.521° | 3.77926 Å | 5.9% |
| 23.824° | 3.73192 Å | 0.2% |
| 24.149° | 3.68236 Å | 1.0% |
| 24.604° | 3.61531 Å | 1.6% |
| 24.968° | 3.56338 Å | 11.1% |
| 25.176° | 3.53453 Å | 8.9% |
| 25.730° | 3.45958 Å | 16.1% |
| 26.681° | 3.33846 Å | 4.0% |
| 27.227° | 3.27267 Å | 2.3% |
| 27.703° | 3.21750 Å | 0.5% |
| 28.142° | 3.16833 Å | 5.3% |
| 28.640° | 3.11437 Å | 1.8% |
| 29.534° | 3.02207 Å | 2.6% |
| 29.993° | 2.97689 Å | 0.7% |
| 30.282° | 2.94911 Å | 0.8% |
| 30.772° | 2.90325 Å | 0.6% |
| 31.307° | 2.85487 Å | 0.3% |
| 31.803° | 2.81146 Å | 2.4% |
| 32.096° | 2.78649 Å | 0.9% |
| 32.604° | 2.74424 Å | 0.5% |
| 33.412° | 2.67967 Å | 2.9% |
| 34.057° | 2.63036 Å | 0.7% |
| 34.602° | 2.59020 Å | 0.6% |
| 35.008° | 2.56108 Å | 1.2% |
| 35.761° | 2.50887 Å | 0.3% |
| 36.179° | 2.48085 Å | 0.8% |
| 37.367° | 2.40462 Å | 1.0% |
| 37.903° | 2.37187 Å | 2.2% |
| 39.081° | 2.30300 Å | 0.7% |

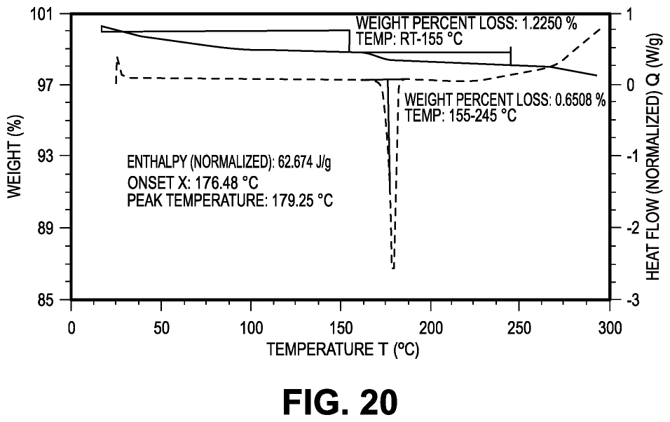

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide exhibiting the TGA pattern as shown in FIG. 20. In some embodiments, the crystalline form exhibits less than 2.9%±0.5 weight loss up to 245° C.±10.0 as determined by thermogravimetric analysis. In some embodiments, the crystalline form exhibits less than 1.9%±0.5 weight loss up to 245° C.±10.0 as determined by thermogravimetric analysis. In some embodiments, the crystalline form exhibits at least 0.9%±0.5 weight loss up to 245° C.±10.0 as determined by thermogravimetric analysis.

Provided herein is crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide wherein the DSC is characterized by an endothermic peak at 179.3° C.±5.0 as shown in FIG. 20.

In some embodiments, provided herein is a composition wherein the crystalline Form IX is substantially free of other crystalline or amorphous forms. In some embodiments, the amount of other crystalline or amorphous forms is 20% (w/w) or less. In some embodiments, the amount of other crystalline or amorphous forms is 15% (w/w) or less. In some embodiments, the amount of other crystalline or amorphous forms is 10% (w/w) or less. In some embodiments, the amount of other crystalline or amorphous forms is 5% (w/w) or less. In some embodiments, the amount of other crystalline or amorphous forms is 1% (w/w) or less.

In some embodiments, provided herein is a composition wherein the crystalline Form IX is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Amorphous form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

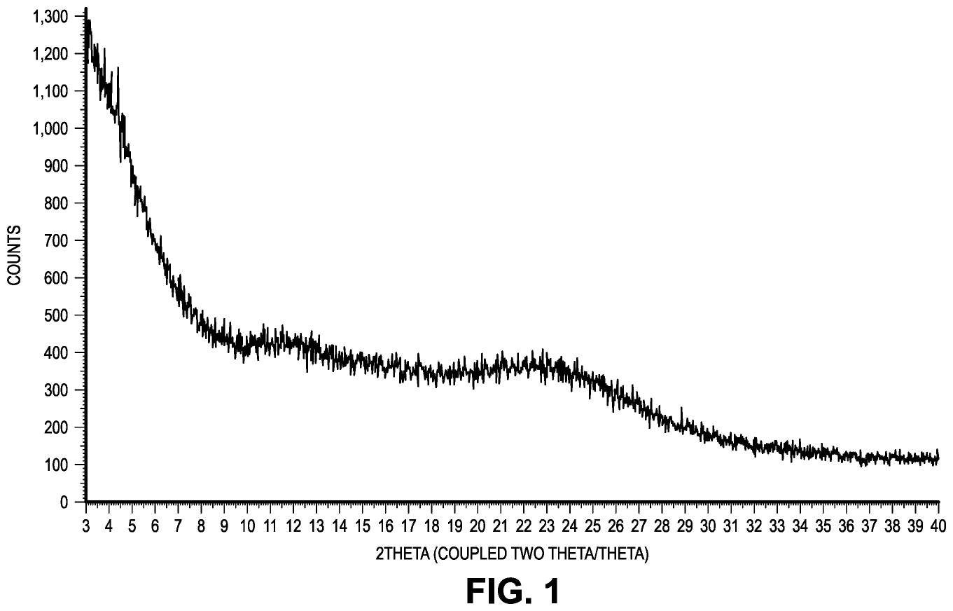

In some embodiments, the present invention provides an amorphous solid state of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, also known as Compound 1. In some embodiments, the amorphous solid state of Compound 1 exhibits the X-ray powder diffraction pattern substantially similar to that shown in FIG. 1.

In some embodiments, provided herein is a composition wherein the amorphous solid state of Compound 1 is substantially free of crystalline forms. In some embodiments, the amount of crystalline forms is 20% (w/w) or less. In some embodiments, the amount of crystalline forms is 15% (w/w) or less. In some embodiments, the amount of crystalline forms is 10% (w/w) or less. In some embodiments, the amount of crystalline forms is 5% (w/w) or less. In some embodiments, the amount of crystalline forms is 1% (w/w) or less.

In some embodiments, provided herein is a composition wherein the amorphous solid state of Compound 1 is substantially free of impurities. In some embodiments, the amount of impurities is 20% (w/w) or less. In some embodiments, the amount of impurities is 15% (w/w) or less. In some embodiments, the amount of impurities is 10% (w/w) or less. In some embodiments, the amount of impurities is 5% (w/w) or less. In some embodiments, the amount of impurities is 2% (w/w) or less. In some embodiments, the amount of impurities is 1% (w/w) or less. In some embodiments, the amount of impurities is 0.5% (w/w) or less. In some embodiments, the amount of impurities is 0.1% (w/w) or less. In some embodiments, the amount of impurities is 0.01% (w/w) or less.

### Pharmaceutical Compositions

In certain embodiments, Compound 1 is administered as a pure chemical. In other embodiments, Compound 1, is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one of Compound 1, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and Compound 1.

One embodiment provides a method of preparing a pharmaceutical composition comprising mixing Compound 1, and a pharmaceutically acceptable carrier.

In certain embodiments, Compound 1, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the formulation comprises a Compound 1; a pharmaceutically acceptable carrier; and a disintegrating agent. In some embodiments, the disintegrating agent is selected from the group consisting of natural starch, a pregelatinized starch, a sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, or a gum. In some embodiments, the disintegrating agent is croscarmellose sodium.

One embodiment provides a pharmaceutical composition comprising a pharmaceutically acceptable excipient and the amorphous form of Compound 1.

One embodiment provides a pharmaceutical composition comprising a pharmaceutically acceptable excipient and the crystalline form I of Compound 1.

One embodiment provides a pharmaceutical composition comprising a pharmaceutically acceptable excipient and the crystalline form II of Compound 1.

One embodiment provides a pharmaceutical composition comprising a pharmaceutically acceptable excipient and the crystalline form III of Compound 1.

One embodiment provides a pharmaceutical composition comprising a pharmaceutically acceptable excipient and the crystalline form IV of Compound 1.

One embodiment provides a pharmaceutical composition comprising a pharmaceutically acceptable excipient and the crystalline form V of Compound 1.

One embodiment provides a pharmaceutical composition comprising a pharmaceutically acceptable excipient and the crystalline form VI of Compound 1.

One embodiment provides a pharmaceutical composition comprising a pharmaceutically acceptable excipient and the crystalline form VII of Compound 1.

One embodiment provides a pharmaceutical composition comprising a pharmaceutically acceptable excipient and the crystalline form VIII of Compound 1.

One embodiment provides a pharmaceutical composition comprising a pharmaceutically acceptable excipient and the crystalline form IX of Compound 1.

The dose of the composition comprising Compound 1, differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides Compound 1, for use in a method of treatment of the human or animal body.

One embodiment provides Compound 1, for use in a method of treatment of cancer or neoplastic disease.

One embodiment provides a use of Compound 1, in the manufacture of a medicament for the treatment of cancer or neoplastic disease.

In some embodiments, described herein is a method of treating cancer in a patient in need thereof comprising administering to the patient Compound 1. In some embodiments, described herein is a method of treating cancer in a patient in need thereof comprising administering to the patient a pharmaceutical composition comprising Compound 1, and a pharmaceutically acceptable excipient.

In some embodiments, described herein is a method of treating cancer in a patient in need thereof comprising administering to the patient the amorphous form of Compound 1. In some embodiments, described herein is a method of treating cancer in a patient in need thereof comprising administering to the patient a pharmaceutical composition comprising the amorphous form of Compound 1, and a pharmaceutically acceptable excipient.

In some embodiments, described herein is a method of treating cancer in a patient in need thereof comprising administering to the patient the crystalline form I of Compound 1. In some embodiments, described herein is a method of treating cancer in a patient in need thereof comprising administering to the patient a pharmaceutical composition comprising the crystalline form I of Compound 1, and a pharmaceutically acceptable excipient.

In some embodiments, described herein is a method of treating cancer in a patient in need thereof comprising administering to the patient the crystalline form II of Compound 1. In some embodiments, described herein is a method of treating cancer in a patient in need thereof comprising administering to the patient a pharmaceutical composition comprising the crystalline form II of Compound 1, and a pharmaceutically acceptable excipient.

In some embodiments, described herein is a method of treating cancer in a patient in need thereof comprising administering to the patient the crystalline form III of Compound 1. In some embodiments, described herein is a method of treating cancer in a patient in need thereof comprising administering to the patient a pharmaceutical composition comprising the crystalline form III of Compound 1, and a pharmaceutically acceptable excipient.

In some embodiments, described herein is a method of treating cancer in a patient in need thereof comprising administering to the patient the crystalline form IV of Compound 1. In some embodiments, described herein is a method of treating cancer in a patient in need thereof comprising administering to the patient a pharmaceutical composition comprising the crystalline form IV of Compound 1, and a pharmaceutically acceptable excipient.

In some embodiments, described herein is a method of treating cancer in a patient in need thereof comprising administering to the patient the crystalline form V of Compound 1. In some embodiments, described herein is a method of treating cancer in a patient in need thereof comprising administering to the patient a pharmaceutical composition comprising the crystalline form V of Compound 1, and a pharmaceutically acceptable excipient.

In some embodiments, described herein is a method of treating cancer in a patient in need thereof comprising administering to the patient the crystalline form VI of Compound 1. In some embodiments, described herein is a method of treating cancer in a patient in need thereof comprising administering to the patient a pharmaceutical composition comprising the crystalline form VI of Compound 1, and a pharmaceutically acceptable excipient.

In some embodiments, described herein is a method of treating cancer in a patient in need thereof comprising administering to the patient the crystalline form VI of Compound 1. In some embodiments, described herein is a method of treating cancer in a patient in need thereof comprising administering to the patient a pharmaceutical composition comprising the crystalline form VI of Compound 1, and a pharmaceutically acceptable excipient.

In some embodiments, described herein is a method of treating cancer in a patient in need thereof comprising administering to the patient the crystalline form VII of Compound 1. In some embodiments, described herein is a method of treating cancer in a patient in need thereof comprising administering to the patient a pharmaceutical composition comprising the crystalline form VII of Compound 1, and a pharmaceutically acceptable excipient.

In some embodiments, described herein is a method of treating cancer in a patient in need thereof comprising administering to the patient the crystalline form VIII of Compound 1. In some embodiments, described herein is a method of treating cancer in a patient in need thereof comprising administering to the patient a pharmaceutical composition comprising the crystalline form VIII of Compound 1, and a pharmaceutically acceptable excipient.

In some embodiments, described herein is a method of treating cancer in a patient in need thereof comprising administering to the patient the crystalline form IX of Compound 1. In some embodiments, described herein is a method of treating cancer in a patient in need thereof comprising administering to the patient a pharmaceutical composition comprising the crystalline form IX of Compound 1, and a pharmaceutically acceptable excipient.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### Examples

The present disclosure is further illustrated by the following examples, which should not be construed as limiting in any way. The experimental procedures to generate the data shown are discussed in more detail below. The disclosure has been described in an illustrative manner, and it is to be understood that the terminology used is intended to be in the nature of description rather than of limitation.

### General Experimental, Instrument, and Methodology Details

A general synthesis for 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide is described in paragraphs in PCT/US2020/057132.

### X-Ray Powder Diffraction (XRPD)

For XRPD analysis, a Bruker D8 Advance X-ray powder diffractometer was used equipped with a LynxEye detector. The XRPD parameters used are listed in Table 10.

TABLE 10
Parameters for XRPD experiments

| | |
|---|---|
| Instrument | Bruker, D8 Advance |
| Radiation | Cu Kα (λ = 1.5418 Å) |
| Detector | LynxEye |
| Scan angle | 3-40° (2θ) |
| Scan step | 0.02° (2θ) |
| Scan speed | 0.2 s/step |
| Tube voltage/current | 40 kV/40 mA |
| Divergence slit | 0.6 mm |
| Rotation | On |
| Sample holder | Zero-background sample pan |

### Differential Scanning Calorimetry (DSC)

DSC was performed using a Discovery DSC 250 (TA Instruments, US). The sample was placed into an aluminum pin-hole hermetic pan and the weight was accurately recorded. The sample was heated at a rate of 10° C./min from 25° C. to the final temperature. The DSC parameters used are listed in Table 11.

TABLE 11
Parameters for DSC experiments

| | |
|---|---|
| Instrument | TA, Discovery DSC 250 |
| Sample pan | Aluminum, pin-holed |
| Temperature range | 25-300° C. |
| Heating rate | 10° C./min |
| Purge gas | N2 |
| Flow rate | 50 mL/min |

### Thermo-Gravimetric Analysis (TGA)

TGA was carried out on a Discovery TGA 55 (TA Instruments, US). The sample was placed into an open tared aluminum pan, automatically weighed, and inserted into the TGA furnace. The sample was heated at a rate of 10° C./min from ambient temperature to the final temperature. The TGA parameters used are listed in Table 12.

TABLE 12
Parameters for TGA experiments

| | |
|---|---|
| Instrument | TA, Discovery TGA 55 |
| Sample pan | Aluminum, open |
| Temperature range | RT-300° C. |
| Heating rate | 10° C./min |
| Purge gas | N2 |
| Flow rate | Balance chamber: 40 mL/min |
| | Sample chamber: 25 mL/min |

### Dynamic Vapor Sorption (DVS)

Moisture sorption/desorption data was collected on a DVS Intrinsic PLUS (SMS, UK). The sample was placed into a tared sample chamber and automatically weighed. The sample was dried at 40° C./0% RH until the dm/dt was less than 0.002% and cooled to 25° C. The DVS parameters used are listed in Table 13.

TABLE 13
Parameters for DVS experiments

| Instrument | SMS, DVS Intrinsic PLUS | |
|---|---|---|
| dm/dt | 0.002%/min | |
| Drying/ Measurement temperature | 40° C./25° C. | |
| Cycle | Full cycle | |
| Save data rate | 5 | s |
| Total flow rate | 200 | ccm |
| Post experiment total flow | 200 | ccm |
| Minimum time per step | 30 | min |
| Maximum time per step | 120 | min |

| | |
|---|---|
| Method | Adsorption: 0, 10, 20, 30, 40, |
| | 50, 60, 70, 80, 90 |
| | Desorption: 80, 70, 60, 50, |
| | 40, 30, 20, 10, 0 |

### Polarized Light Microscopy (PLM)

Light microscopy was performed using a Polarizing Microscope ECLIPSE LV100POL (Nikon, JPN). A small amount of sample was placed on a glass slide, with or without immersion oil, and covered with a glass slip. The sample was observed by microscope.

### Proton Nuclear Magnetic Resonance (1H-NMR)

1H-NMR spectra were collected on a Bruker 400 MHz instrument. Unless specified, samples were prepared in DMSO-d6 solvent. The data was analyzed using MestReNova.

### High Performance Liquid Chromatography (HPLC) Method

HPLC analysis was performed with an Agilent HPLC 1260 series instrument. HPLC method for solubility and stability testing is listed in Table 14.

TABLE 14
Parameters for HPLC experiments

| Instrument | Agilent 1260 series | | |
|---|---|---|---|
| Column | Ascentis Express C18, | | |
| | 4.6*100 mm, 2.7 μm | | |
| Column temperature | 40° C. | | |
| Mobile phase | A: 0.1% TFA in | | |
| | H2OB: 0.1% TFA in ACN | | |
| Flow rate | 1.0 mL/min | | |
| Injection volume | 4 μL | | |
| Detector & Wavelength | DAD; 225 nm | | |
| Run time | 12 min | | |
| Post time | 3 min | | |
| Diluent | ACN/water (1:1) | | |
| | Time (min) | % A | % B |
| Gradient | 0.0 | 80 | 20 |
| | 6.0 | 30 | 70 |
| | 8.0 | 0 | 100 |

### Example 1: Synthesis of Crystalline Form I of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form I was initially identified from slurry experiments in water at 50° C., and later obtained from drop-solvent grinding in water and anti-solvent precipitation in aqueous systems, including MeOH/water, EtOH/water, acetone/water, ACN/water and THF/water. 3.87% of weight loss attributed to water was observed by TGA, and the endothermic peak from RT—76° C. attributed to dehydration was observed by DSC. The theoretical water content of 1 mol water is 3.3%, suggesting that Form I is a mono-hydrate. DSC also exhibited two melting peaks at 104° C. and 194° C. after dehydration, which corresponded to melting of Form V and Form II, respectively.

### Example 2: Synthesis of Crystalline Form II of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form II was obtained from slurry experiments in many solvents, including MEK and IPAC at RT, and MTBE, MEK, toluene, IPA, EA at 50° C. Form II was also prepared by anti-solvent precipitation in THF/n-heptane or acetone/MTBE, and slow cooling crystallization form EA. No weight loss was observed by TGA and DSC exhibited an endothermic peak at 191° C. which was due to melting. Therefore, Form II is an anhydrate. DVS result showed Form II was slightly hygroscopic with 0.47% of water uptake at 90% RH and the crystal form remained unchanged after DVS testing.

### Example 3: Synthesis of Crystalline Form III of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form III was obtained from slurry experiments in n-heptane at 50° C. Form III is an anhydrate with a melting point at 155° C. and hygroscopicity. About 1.9% of weight loss at RT—205° C. in two steps was observed by TGA, which were attributed to adsorbed water. DSC showed the melting peak was closely followed by a phase transition peak at 161° C., and a melting peak at 191° C., suggesting that Form III converts to Form II during heating.

### Example 4: Synthesis of Crystalline Form IV of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form IV was obtained from slurry experiments in water at RT for 3 days. 1.8% of weight loss at RT—94° C. in two steps and a broad endothermic peak from 41-66° C. due to dehydration were observed by TGA and DSC, respectively. The theoretical water content of 0.5 mol is 1.65%, suggesting Form IV was a hemi-hydrate. Based on the DSC data, Form IV may convert to Form V after dehydration, and then convert to Form II during heating.

### Example 5: Synthesis of Crystalline Form V of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form V can be obtained by dehydrating Form I. Form V is an anhydrate with a melting point at 102° C. and hygroscopicity. About 1% of weight loss at RT—100° C. was observed in TGA, due to loss of adsorbed water.

### Example 6: Synthesis of Crystalline Form VI of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form VI was generated from slurry studies in toluene at RT. Based on TGA, DSC and 1H NMR data, Form VI is a toluene solvate (3.5% Toluene by NMR). After desolvation, Form VI converts to Form II during heating by DSC.

### Example 7: Synthesis of Crystalline Form VII of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form VII was identified from slurry studies in IPA at RT. Form VII is an IPA solvate with 2.9% of weight loss at RT—140° C. in TGA, attributed to 2.8% IPA by NMR. The theoretical IPA content of 1 mol IPA was 10.3%. After desolvation, Form VII converts to Form II during heating.

### Example 8: Synthesis of Crystalline Form VIII of 1-(3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form VIII was obtained from IPA by fast cooling crystallization. Similar to Form VII, Form VIII may be also a solvate of IPA with 6.3% of weight loss at RT—140° C. in two steps in TGA, mainly due to loss of 5% IPA by NMR. The theoretical IPA content of 1 mol IPA was 10.3%, suggesting Form VIII is a hemi-IPA solvate. After desolvation, Form VIII would also convert to Form II during heating.

### Example 9: Synthesis of Crystalline Form IX of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Form IX was precipitated from EA at 60° C. TGA showed 1.8% of weight loss at RT—245° C. in two steps attributed to the adsorbed water and residual solvents, respectively, based on the 1H-NMR data. DSC exhibited a sharp endothermic peak at 176° C. due to melting, suggesting Form IX is an anhydrate containing residual solvent which is released during the melting.

### Example 10: Synthesis of the Amorphous form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide (Compound 1

Slow evaporation performed in ACN, acetone, methanol, MEK, ethanol, ethyl acetate, and isopropyl alcohol yielded amorphous Compound 1.

### Example 10: Characterization and Comparison of the Solid Forms of 1-((3S,5R)-1-acryloyl-5-(methoxymethyl)pyrrolidin-3-yl)-3-((1-cyclopropyl-4,6-difluoro-1H- benzo[d]imidazol-5-yl)ethynyl)-5-(methylamino)-1H-pyrazole-4-carboxamide (Compound 1

A summary of the characterization data for the solid forms of Compound 1 identified is shown below in Table 15. FIG. 21 shows conversion map of the solid state forms of Compound 1.

TABLE 15
Summary of characterization data

| Form | DSC, Onset | TGA | ¹H-NMR | |
|---|---|---|---|---|
| Solvation | (° C.) (ΔH, J/g) | % wt. loss/@T | Residual solvent | Comment |
| Form I | 26 (102) | 3.9//RT-76° C. | Not detected | Obtained by |
| Mono-hydrate | 102 (12.5) | | | slurryin water |
| | 193 (0.99) | | | |
| Form II | 191 (66) | ~0/RT-200° C. | Not detected | Obtained in most |
| Anhydrate | | | | conditions, slightly |
| | | | | hygroscopic with0.47% |
| | | | | weight gain at 90% RH |
| Form III | 155 (6.1) | 0.5/RT-59° C. | Not detected | Hygroscopic |
| Anhydrate | 161 (13.4, exo) | 1.36/59-205° C. | | |
| | 191 (47.2) | | | |
| Form IV | 41 (93.7), | 0.85/RT-52° C. | Not detected | Obtained by slurryin |
| Hemi-hydrate | 102 (10.2) | 1.0/52-94° C. | | water at RT, |
| | 194 (0.9) | | | unrepeatable |
| Form V | 102 (13) | 1.1/RT-101° C. | Not detected | Dehydrated Form I |
| Anhydrate | | | | |
| Form VI | 107 (22.5) | 0.6/RT-61° C. | 3.5% of toluene | Obtained by slurryin |
| Toluene | 138 (31.5, exo) | 3.65/61-128° C. | | toluene at RT |
| solvate | 190 (36) | | | |
| Form VII | 113 (29, broad) | 2.8/RT-119° C. | 2.8% of IPA | Obtained by slurryin |
| IPA solvate | 151 (30, exo) | | | IPA at RT |
| | 193 (35) | | | |
| Form VIII | 106 (53) | 3.3/RT-103° C. | 5% of IPA | Fast cooling in IPA |
| IPA Solvate | 154 (10, exo) | 3.1/61-140° C. | | |
| | 194 (16) | | | |
| Form IX | 176 (62.7) | 1.23/RT-155° C. | 0.6% of EA | Obtained from EAat 60° C. |
| Anhydrate | | 0.65/155-245° C. | | |

### Solubility Testing

Preliminary solubility of Compound 1 (Form V) was estimated in 14 selected solvents at RT using solvent addition method via visual assessment of samples. The results are summarized in Table 16. The material showed relatively high solubility in MeOH, THF, acetone, MEK and ACN (>20 mg/mL), while practically insoluble (<1 mg/mL) in non-polar solvents and water.

TABLE 16
Solubility of Compound 1

| | Solubility | | Solubility |
|---|---|---|---|
| Solvent | (mg/mL) | Solvent | (mg/mL) |
| MeOH | >106 | Isobutanol | ~6 |
| THF | >100 | IPA | ~5 |
| Acetone | ~34 | IPAC | ~4 |
| MEK | ~31 | Toluene | <1 |
| ACN | >27, <36 | MTBE | <0.75 |
| EtOH | ~21 | Water | <1 |
| EA | ~8 | n-Heptane | <1 |

### Slurry Studies

Slurry studies were carried out with Form V as the starting material in many solvents at RT for 3 days and 50° C. for 1 day. The results are summarized in Table 17. Form II was obtained from most of the solvents. Form I and Form III were obtained from water and heptane at 50° C., respectively. Form IV, Form VI, and Form VII was obtained from water, toluene and IPA at RT, respectively.

TABLE 17
Slurry Studies

| | | Slurry | |
|---|---|---|---|
| | | Conc. | XRPD |
| Temp. | Solvent | (mg/mL) | Result |
| 50° C. | Water | 30 | Form I |
| | MTBE | 30 | Form II |
| | Heptane | 30 | Form III |
| | Isobutanol | 34 | Jelly |
| | MEK | 70 | Form II |
| | IPAC | 50 | Form II + |
| | | | Form III |
| | Toluene | 50 | Form II |
| | IPA | 50 | Form II |
| | EA | 50 | Form II |
| RT | Water | 30 | Form IV |
| | MTBE | 30 | Nearly |
| | | | amorphous |
| | Heptane | 30 | Form IV + V |
| | Isobutanol | 50 | Jelly |
| | MEK | 70 | Form II |
| | IPAC | 50 | Form II |
| | Toluene | 50 | Form VI |
| | IPA | 50 | Form VII |
| | EA | 50 | Jelly |

### Inter-Conversion Studies

Competitive slurries of Form I, II and V were performed in IPAC at RT and 50° C. The results showed that each mixture completely converted to Form II after 3 days. A competitive slurry of Form III and Form II was conducted in IPA and IPAC at RT and 50° C., respectively, and all mixtures converted to Form II after 1 day. A competitive slurry of Form II and IX was also performed in IPA and IPAC at RT and 50° C. The results showed the mixture completely convert to Form II in IPA at 50° C. after 1 day. In IPA at RT and IPA and EA at RT and 50° C., all mixtures tended to convert to Form II. Form II was found to be the most stable form in non-aqueous solvents.

### Water Activity Studies

Water activity studies of Form I, II and V were also performed in IPA/water with different water content at RT and 50° C. The results showed Form II was physically stable at aw≤0.35 for RT and aw≤0.55 for 50° C., while Form I was more stable than Form II at aw≥0.75 for 50° C. and aw≥0.55 for RT. Form II was found to be the more stable form in non-aqueous solvents, and also physically stable at aw≤0.35 for RT and aw≤0.55 for 50° C.

### Stability Studies

The solid stability of Form II was evaluated at 60° C. for 7 days, 40° C./75% RH for 6 days, and RT/92.5% RH for 2 days, respectively. Form II was physically and chemically stable at 40° C./75% RH for 6 days. The purity of Compound 1 decreased by about 0.3% at 60° C. for 3 days and 7 days, mainly due to the increase of the impurities at RRT 0.9, 0.91 and 1.07. Form II was physically stable in high humidity condition for 2 days.

### Drug Product Formulation and Manufacturing Process

The formulation of the drug product provides an immediate release of Compound 1 over a period of approximately 1 hr. As the tablet is exposed to water and starts to disintegrate, drug substance is quickly released from the tablet core. The tablets are intended to dissolve completely in the stomach where the solubility is highest. In order to enable fast dissolution, a super-disintegrant, such as croscarmellose sodium, is added to the formulation. Other components of the formulation include fillers such as microcrystalline cellulose, and mannitol, glidant such as silicon dioxide, and a lubricant such as magnesium stearate. The tablets are film-coated using a non-functional coating containing polymer such as polyvinyl alcohol, plasticizer such as PEG, titanium dioxide, and other coloring pigments as needed.

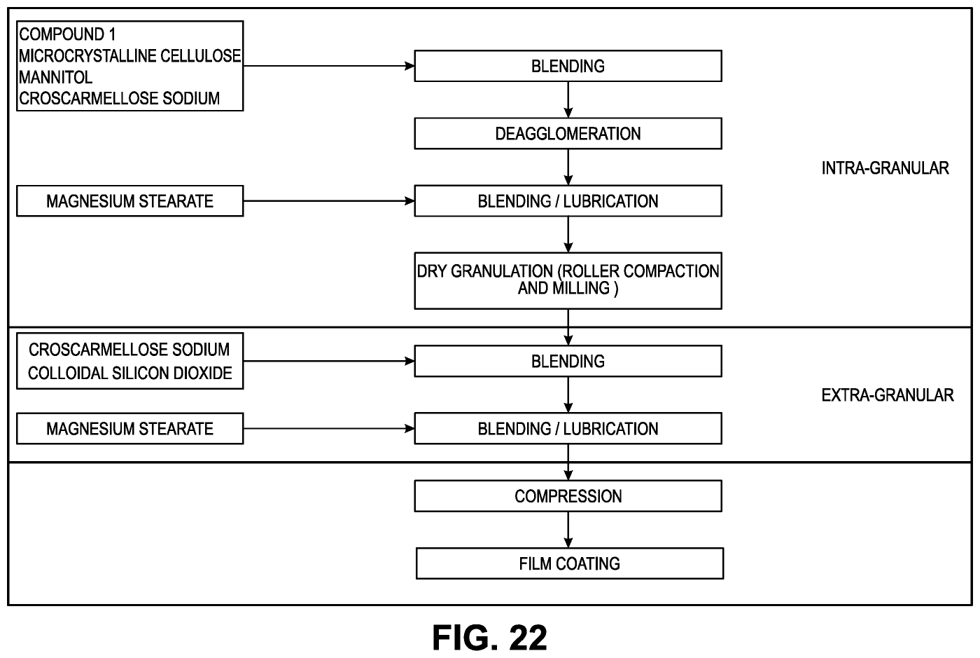

FIG. 22 illustrates the manufacturing process used for an exemplary drug product. A dry granulation process was selected for the manufacture of tablets in order to improve blend flow in the tablet press and weight uniformity of the tablet core formulation. The tablet manufacturing process consists of first blending Compound 1, microcrystalline cellulose, mannitol, and croscarmellose sodium in a blender. The blended material is passed through a Comil to breakup any aggregates, before adding magnesium stearate and blending further. The lubricated blend is dry granulated by a process such as roller compaction to increase density of the material, followed by milling. To the granulated milled material, are added croscarmellose sodium, silicon dioxide, and magnesium stearate. This mixture of granules and extra-granular excipients is blended to prepare the final composition for tableting. Tablet cores are compressed using a rotary tablet press. After compression, tablet cores are coated using an aqueous based film coating system in a pan coater.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A solid form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, depicted below as Compound 1,

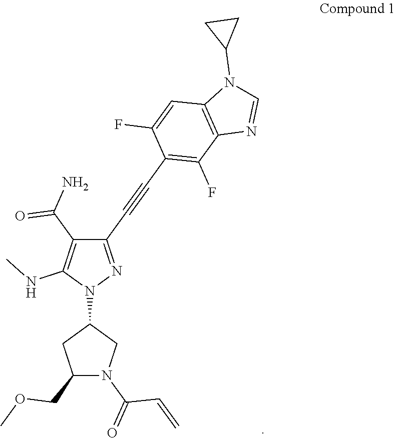 [SMILES: C=CC(=O)N1C[C@@H](n2nc(C#Cc3c(F)cc4c(ncn4C4CC4)c3F)c(C(N)=O)c2NC)C[C@@H]1COC]

2. A solid form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide, wherein the solid form is crystalline.

3. The solid form of claim 2, wherein the solid form exhibits an X-ray powder diffraction reflection at a 2-theta value of 11.8°±0.3.

4. The solid form of claim 3, wherein the solid form exhibits an X-ray powder diffraction reflection at a 2-theta value of 7.9°±0.3 and 13.7°±0.3.

5. The solid form of any one of claim 3 or 4, wherein the solid form exhibits an X-ray powder diffraction reflection at a 2-theta value of 9.5°±0.3, 18.9°±0.3, and 26.8°±0.3.

6. The solid form of any one of claims 3 to 5, wherein the solid form exhibits an X-ray powder diffraction reflection at a 2-theta value of 15.9°±0.3, 20.5°±0.3, and 22.3°±0.3.

7. The solid form of any one of claims 3 to 6, wherein the solid form exhibits an X-ray powder diffraction reflection at a 2-theta value of 20.8°±0.3, 25.7°±0.3, and 26.3°±0.3.

8. The solid form of claim 2, wherein the solid form exhibits at least one X-ray powder diffraction reflection selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

9. The solid form of claim 8, wherein the solid form exhibits at least two X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

10. The solid form of claim 9, wherein the solid form exhibits at least three X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

11. The solid form of claim 10, wherein the solid form exhibits at least four X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

12. The solid form of claim 11, wherein the solid form exhibits at least five X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

13. The solid form of claim 12, wherein the solid form exhibits at least six X-ray powder diffraction reflections selected from 7.9°±0.3, 9.5°±0.3, 11.8°±0.3, 13.7°±0.3, 15.9°±0.3, 18.9°±0.3, 20.5°±0.3, 20.8°±0.3, 22.3°±0.3, 25.7°±0.3, and 26.3°±0.3.

14. The solid form of claim 2, wherein the solid form exhibits the X-ray powder diffraction pattern as shown in FIG. 4.

15. The solid form of any one of claims 3 to 14, wherein the solid form exhibits a differential scanning calorimetry thermogram comprising an endothermic peak at 193.1° C.±5.0.

16. The solid form of any one of claims 3 to 15, wherein the solid form exhibits the differential scanning calorimetry thermogram as shown in FIG. 5.

17. The solid form of any one of claims 3 to 16, wherein the solid form exhibits less than 1.1%±0.5 weight loss up to 200° C.±10.0 as determined by thermogravimetric analysis.

18. The solid form of any one of claims 3 to 17, wherein the solid form exhibits the thermogravimetric analysis thermogram as shown in FIG. 5.

19. The solid form of any one of claims 3 to 18, wherein the solid form is substantially free of other crystalline or amorphous forms.

20. The solid form of claim 19, wherein the amount of other crystalline or amorphous forms is 5% (w/w) or less.

21. The solid form of any one of claims 3 to 20, wherein the solid form is substantially free of impurities.

22. The solid form of claim 21, wherein the amount of impurities is 2% or less.

23. The solid form of claim 2, wherein the solid form exhibits an X-ray powder diffraction reflection at a 2-theta value of 5.0°±0.3.

24. The solid form of claim 23, wherein the solid form exhibits an X-ray powder diffraction reflection at a 2-theta value of 10.1°±0.3 and 17.1°±0.3.

25. The solid form of any one of claim 23 or 24, wherein the solid form exhibits an X-ray powder diffraction reflection at a 2-theta value of 8.5°±0.3, 24.8°±0.3, and 25.8°±0.3.

26. The solid form of any one of claims 23 to 25, wherein the solid form exhibits an X-ray powder diffraction reflection at a 2-theta value of 9.3°±0.3, 13.0°±0.3, and 16.4°±0.3.

27. The solid form of any one of claims 23 to 26, wherein the solid form exhibits an X-ray powder diffraction reflection at a 2-theta value of 12.1°±0.3 and 15.2°±0.3.

28. The solid form of claim 2, wherein the solid form exhibits at least one X-ray powder diffraction reflection selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

29. The solid form of claim 28, wherein the solid form exhibits at least two X-ray powder diffraction reflections selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

30. The solid form of claim 29, wherein the solid form exhibits at least three X-ray powder diffraction reflections selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

31. The solid form of claim 30, wherein the solid form exhibits at least four X-ray powder diffraction reflections selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

32. The solid form of claim 31, wherein the solid form exhibits at least five X-ray powder diffraction reflections selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

33. The solid form of claim 32, wherein the solid form exhibits at least six X-ray powder diffraction reflections selected from 5.0°±0.3, 8.5°±0.3, 9.3°±0.3, 10.1°±0.3, 12.1°±0.3, 13.0°±0.3, 15.2°±0.3, 16.4°±0.3, 17.1°±0.3, 24.8°±0.3, and 25.8°±0.3.

34. The solid form of claim 2, wherein the solid form exhibits the X-ray powder diffraction pattern as shown in FIG. 2.

35. The solid form of any one of claims 23 to 34, wherein the solid form exhibits a differential scanning calorimetry thermogram comprising endothermic peaks at 53.5° C.±10.0, 106.5° C.±5.0, and 193.7° C.±5.0.

36. The solid form of any one of claims 23 to 35, wherein the solid form exhibits the differential scanning calorimetry thermogram as shown in FIG. 3.

37. The solid form of any one of claims 23 to 36, wherein the solid form exhibits less than 4.9%±0.5 weight loss up to 76° C.±10.0 as determined by thermogravimetric analysis.

38. The solid form of any one of claims 23 to 37, wherein the solid form exhibits the thermogravimetric analysis thermogram as shown in FIG. 3.

39. The solid form of any one of claims 23 to 38, wherein the solid form is substantially free of other crystalline or amorphous forms.

40. The solid form of claim 39, wherein the amount of other crystalline or amorphous forms is 5% (w/w) or less.

41. The solid form of any one of claims 23 to 40, wherein the solid form is substantially free of impurities.

42. The solid form of claim 41, wherein the amount of impurities is 2% or less.

43. The solid form of claim 2, wherein the solid form exhibits an X-ray powder diffraction reflection at a 2-theta value of 8.7°±0.3.

44. The solid form of claim 43, wherein the solid form exhibits an X-ray powder diffraction reflection at a 2-theta value of 5.0°±0.3 and 12.8°±0.3.

45. The solid form of any one of claim 43 or 44, wherein the solid form exhibits an X-ray powder diffraction reflection at a 2-theta value of 23.9°±0.3 and 24.3°±0.3.

46. The solid form of any one of claims 43 to 45, wherein the solid form exhibits an X-ray powder diffraction reflection at a 2-theta value of 11.5°±0.3, 20.3°±0.3, and 21.2°±0.3.

47. The solid form of any one of claims 43 to 46, wherein the solid form exhibits an X-ray powder diffraction reflection at a 2-theta value of 11.6°±0.3, 15.9°±0.3, and 25.8°±0.3.

48. The solid form of claim 2, wherein the solid form exhibits at least one X-ray powder diffraction reflection selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

49. The solid form of claim 48, wherein the solid form exhibits at least two X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

50. The solid form of claim 49, wherein the solid form exhibits at least three X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

51. The solid form of claim 50, wherein the solid form exhibits at least four X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

52. The solid form of claim 51, wherein the solid form exhibits at least five X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

53. The solid form of claim 52, wherein the solid form exhibits at least six X-ray powder diffraction reflections selected from 5.0°±0.3, 8.7°±0.3, 11.5°±0.3, 11.6°±0.3, 12.8°±0.3, 15.8°±0.3, 20.3°±0.3, 21.2°±0.3, 23.9°±0.3, 24.3°±0.3, and 25.8°±0.3.

54. The solid form of claim 2, wherein the solid form exhibits the X-ray powder diffraction pattern as shown in FIG. 7.

55. The solid form of any one of claims 43 to 54, wherein the solid form exhibits a differential scanning calorimetry thermogram comprising endothermic peaks at 164.0° C.±5.0 and 190.6° C.±5.0.

56. The solid form of any one of claims 43 to 55, wherein the solid form exhibits the differential scanning calorimetry thermogram as shown in FIG. 8.

57. The solid form of any one of claims 43 to 56, wherein the solid form exhibits less than 2.9%±0.5 weight loss up to 205° C.±10.0 as determined by thermogravimetric analysis.

58. The solid form of any one of claims 43 to 57, wherein the solid form exhibits the thermogravimetric analysis thermogram as shown in FIG. 8.

59. The solid form of any one of claims 43 to 58, wherein the solid form is substantially free of other crystalline or amorphous forms.

60. The solid form of claim 59, wherein the amount of other crystalline or amorphous forms is 5% (w/w) or less.

61. The solid form of any one of claims 43 to 60, wherein the solid form is substantially free of impurities.

62. The solid form of claim 61, wherein the amount of impurities is 2% or less.

63. The solid form of claim 2, wherein the solid form exhibits an X-ray powder diffraction reflection at a 2-theta value of 5.1°±0.3.

64. The solid form of claim 63, wherein the solid form exhibits an X-ray powder diffraction reflection at a 2-theta value of 10.2°±0.3 and 8.6°±0.3.

65. The solid form of any one of claim 63 or 64, wherein the solid form exhibits an X-ray powder diffraction reflection at a 2-theta value of 12.2°±0.3 and 17.3°±0.3.

66. The solid form of any one of claims 63 to 65, wherein the solid form exhibits an X-ray powder diffraction reflection at a 2-theta value of 6.4°±0.3, 13.4°±0.3, and 15.3°±0.3.

67. The solid form of any one of claims 63 to 66, wherein the solid form exhibits an X-ray powder diffraction reflection at a 2-theta value of 15.8°±0.3, and 26.0°±0.3.

68. The solid form of claim 2, wherein the solid form exhibits at least one X-ray powder diffraction reflection selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

69. The solid form of claim 68, wherein the solid form exhibits at least two X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

70. The solid form of claim 69, wherein the solid form exhibits at least three X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

71. The solid form of claim 70, wherein the solid form exhibits at least four X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

72. The solid form of claim 71, wherein the solid form exhibits at least five X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

73. The solid form of claim 72, wherein the solid form exhibits at least six X-ray powder diffraction reflections selected from 5.1°±0.3, 6.4°±0.3, 8.6°±0.3, 10.2°±0.3, 12.2°±0.3, 13.4°±0.3, 15.3°±0.3, 15.8°±0.3, 17.3°±0.3, and 26.0°±0.3.

74. The solid form of claim 2, wherein the solid form exhibits the X-ray powder diffraction pattern as shown in FIG. 9.

75. The solid form of any one of claims 63 to 74, wherein the solid form exhibits a differential scanning calorimetry thermogram comprising endothermic peaks at 66.2° C.±5.0, 106.4° C.±5.0, and 193.6° C.±5.0.

76. The solid form of any one of claims 63 to 75, wherein the solid form exhibits the differential scanning calorimetry thermogram as shown in FIG. 10.

77. The solid form of any one of claims 63 to 76, wherein the solid form exhibits less than 2.9%±0.5 weight loss up to 94° C.±10.0 as determined by thermogravimetric analysis.

78. The solid form of any one of claims 63 to 77, wherein the solid form exhibits the thermogravimetric analysis thermogram as shown in FIG. 10.

79. The solid form of any one of claims 63 to 78, wherein the solid form is substantially free of other crystalline or amorphous forms.

80. The solid form of claim 79, wherein the amount of other crystalline or amorphous forms is 5% (w/w) or less.

81. The solid form of any one of claims 63 to 80, wherein the solid form is substantially free of impurities.

82. The solid form of claim 81, wherein the amount of impurities is 2% or less.

83. The solid form of claim 2, wherein the solid form exhibits an X-ray powder diffraction reflection at a 2-theta value of 5.1°±0.3.

84. The solid form of claim 83, wherein the solid form exhibits an X-ray powder diffraction reflection at a 2-theta value of 10.4°±0.3.

85. The solid form of any one of claim 83 or 84, wherein the solid form exhibits an X-ray powder diffraction reflection at a 2-theta value of 6.4°±0.3 and 25.6°±0.3.

86. The solid form of any one of claims 83 to 85, wherein the solid form exhibits an X-ray powder diffraction reflection at a 2-theta value of 8.7°±0.3, 11.9°±0.3, and 15.6°±0.3.

87. The solid form of any one of claims 83 to 86, wherein the solid form exhibits an X-ray powder diffraction reflection at a 2-theta value of 13.5°±0.3, and 25.8°±0.3.

88. The solid form of claim 2, wherein the solid form exhibits at least one X-ray powder diffraction reflection selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

89. The solid form of claim 88, wherein the solid form exhibits at least two X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

90. The solid form of claim 89, wherein the solid form exhibits at least three X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

91. The solid form of claim 90, wherein the solid form exhibits at least four X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

92. The solid form of claim 91, wherein the solid form exhibits at least five X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

93. The solid form of claim 92, wherein the solid form exhibits at least six X-ray powder diffraction reflections selected from 5.1°±0.3, 6.4°±0.3, 8.7°±0.3, 10.4°±0.3, 11.9°±0.3, 13.5°±0.3, 15.6°±0.3, 25.8°±0.3, and 25.6°±0.3.

94. The solid form of claim 2, wherein the solid form exhibits the X-ray powder diffraction pattern as shown in FIG. 11.

95. The solid form of any one of claims 83 to 94, wherein the solid form exhibits a differential scanning calorimetry thermogram comprising an endothermic peak at 105.8° C.±5.0.

96. The solid form of any one of claims 83 to 95, wherein the solid form exhibits the differential scanning calorimetry thermogram as shown in FIG. 12.

97. The solid form of any one of claims 83 to 96, wherein the solid form exhibits less than 2.1%±0.5 weight loss up to 101° C.±10.0 as determined by thermogravimetric analysis.

98. The solid form of any one of claims 83 to 97, wherein the solid form exhibits the thermogravimetric analysis thermogram as shown in FIG. 12.

99. The solid form of any one of claims 83 to 98, wherein the solid form is substantially free of other crystalline or amorphous forms.

100. The solid form of claim 99, wherein the amount of other crystalline or amorphous forms is 5% (w/w) or less.

101. The solid form of any one of claims 83 to 100, wherein the solid form is substantially free of impurities.

102. The solid form of claim 101, wherein the amount of impurities is 2% or less.

103. The solid form of claim 2, wherein the solid form exhibits an X-ray powder diffraction reflection at a 2-theta value of 9.5°±0.3.

104. The solid form of claim 103, wherein the solid form exhibits an X-ray powder diffraction reflection at a 2-theta value of 5.2°±0.3 and 23.7°±0.3.

105. The solid form of any one of claim 103 or 104, wherein the solid form exhibits an X-ray powder diffraction reflection at a 2-theta value of 5.7°±0.3, 10.2°±0.3, and 19.5°±0.3.

106. The solid form of any one of claims 103 to 105, wherein the solid form exhibits an X-ray powder diffraction reflection at a 2-theta value of 6.5°±0.3, 15.5°±0.3, and 26.8°±0.3.

107. The solid form of any one of claims 103 to 106, wherein the solid form exhibits an X-ray powder diffraction reflection at a 2-theta value of 7.4°±0.3, 15.0°±0.3, 19.0°±0.3, and 24.7°±0.3.

108. The solid form of claim 2, wherein the solid form exhibits at least one X-ray powder diffraction reflection selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

109. The solid form of claim 108, wherein the solid form exhibits at least two X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

110. The solid form of claim 109, wherein the solid form exhibits at least three X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

111. The solid form of claim 110, wherein the solid form exhibits at least four X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

112. The solid form of claim 111, wherein the solid form exhibits at least five X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

113. The solid form of claim 112, wherein the solid form exhibits at least six X-ray powder diffraction reflections selected from 5.2°±0.3, 5.7°±0.3, 6.5°±0.3, 7.4°±0.3, 9.5°±0.3, 10.2°±0.3, 15.0°±0.3, 15.5°±0.3, 19.0°±0.3, 19.5°±0.3, 23.7°±0.3, 24.7°±0.3, and 26.8°±0.3.

114. The solid form of claim 2, wherein the solid form exhibits the X-ray powder diffraction pattern as shown in FIG. 13.

115. The solid form of any one of claims 103 to 114, wherein the solid form exhibits a differential scanning calorimetry thermogram comprising endothermic peaks at 112.3° C.±5.0 and 193.7° C.±5.0.

116. The solid form of any one of claims 103 to 115, wherein the solid form exhibits the differential scanning calorimetry thermogram as shown in FIG. 14.

117. The solid form of any one of claims 103 to 116, wherein the solid form exhibits less than 4.3%±0.5 weight loss up to 126° C.±10.0 as determined by thermogravimetric analysis.

118. The solid form of any one of claims 103 to 117, wherein the solid form exhibits the thermogravimetric analysis thermogram as shown in FIG. 14.

119. The solid form of any one of claims 103 to 118, wherein the solid form is substantially free of other crystalline or amorphous forms.

120. The solid form of claim 119, wherein the amount of other crystalline or amorphous forms is 5% (w/w) or less.

121. The solid form of any one of claims 103 to 120, wherein the solid form is substantially free of impurities.

122. The solid form of claim 121, wherein the amount of impurities is 2% or less.

123. The solid form of claim 2, wherein the solid form exhibits an X-ray powder diffraction reflection at a 2-theta value of 5.4°±0.3.

124. The solid form of claim 123, wherein the solid form exhibits an X-ray powder diffraction reflection at a 2-theta value of 9.8°±0.3 and 23.4°±0.3.

125. The solid form of any one of claim 123 or 124, wherein the solid form exhibits an X-ray powder diffraction reflection at a 2-theta value of 5.0°±0.3 and 15.2°±0.3.

126. The solid form of any one of claims 123 to 125, wherein the solid form exhibits an X-ray powder diffraction reflection at a 2-theta value of 10.1°±0.3, 13.6°±0.3, and 26.7°±0.3.

127. The solid form of any one of claims 123 to 126, wherein the solid form exhibits an X-ray powder diffraction reflection at a 2-theta value of 9.1°±0.3, 25.6°±0.3, and 27.2°±0.3.

128. The solid form of claim 2, wherein the solid form exhibits at least one X-ray powder diffraction reflection selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

129. The solid form of claim 128, wherein the solid form exhibits at least two X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

130. The solid form of claim 129, wherein the solid form exhibits at least three X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

131. The solid form of claim 130, wherein the solid form exhibits at least four X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

132. The solid form of claim 131, wherein the solid form exhibits at least five X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

133. The solid form of claim 132, wherein the solid form exhibits at least six X-ray powder diffraction reflections selected from 5.0°±0.3, 5.4°±0.3, 9.1°±0.3, 9.8°±0.3, 10.1°±0.3, 13.6°±0.3, 15.2°±0.3, 23.4°±0.3, 25.6°±0.3, 26.7°±0.3, and 27.2°±0.3.

134. The solid form of claim 2, wherein the solid form exhibits the X-ray powder diffraction pattern as shown in FIG. 15.

135. The solid form of any one of claims 123 to 134, wherein the solid form exhibits a differential scanning calorimetry thermogram comprising endothermic peaks at 120.8° C.±5.0 and 195.1° C.±5.0.

136. The solid form of any one of claims 123 to 135, wherein the solid form exhibits the differential scanning calorimetry thermogram as shown in FIG. 16.

137. The solid form of any one of claims 123 to 136, wherein the solid form exhibits less than 3.9%±0.5 weight loss up to 119° C.±10.0 as determined by thermogravimetric analysis.

138. The solid form of any one of claims 123 to 137, wherein the solid form exhibits the thermogravimetric analysis thermogram as shown in FIG. 16.

139. The solid form of any one of claims 123 to 138, wherein the solid form is substantially free of other crystalline or amorphous forms.

140. The solid form of claim 139, wherein the amount of other crystalline or amorphous forms is 5% (w/w) or less.

141. The solid form of any one of claims 103 to 140, wherein the solid form is substantially free of impurities.

142. The solid form of claim 141, wherein the amount of impurities is 2% or less.

143. The solid form of claim 2, wherein the solid form exhibits an X-ray powder diffraction reflection at a 2-theta value of 5.8°±0.3.

144. The solid form of claim 143, wherein the solid form exhibits an X-ray powder diffraction reflection at a 2-theta value of 5.2°±0.3 and 15.2°±0.3.

145. The solid form of any one of claim 143 or 144, wherein the solid form exhibits an X-ray powder diffraction reflection at a 2-theta value of 5.0°±0.3 and 10.1°±0.3.

146. The solid form of any one of claims 143 to 145, wherein the solid form exhibits an X-ray powder diffraction reflection at a 2-theta value of 8.4°±0.3, 9.8°±0.3, and 23.4°±0.3.

147. The solid form of any one of claims 143 to 146, wherein the solid form exhibits an X-ray powder diffraction reflection at a 2-theta value of 10.4°±0.3, 14.2°±0.3, and 25.5°±0.3.

148. The solid form of claim 2, wherein the solid form exhibits at least one X-ray powder diffraction reflection selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

149. The solid form of claim 148, wherein the solid form exhibits at least two X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

150. The solid form of claim 149, wherein the solid form exhibits at least three X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

151. The solid form of claim 150, wherein the solid form exhibits at least four X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

152. The solid form of claim 151, wherein the solid form exhibits at least five X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

153. The solid form of claim 152, wherein the solid form exhibits at least six X-ray powder diffraction reflections selected from 5.0°±0.3, 5.2°±0.3, 5.8°±0.3, 8.4°±0.3, 9.8°±0.3, 10.1°±0.3, 10.4°±0.3, 14.2°±0.3, 15.2°±0.3, 23.4°±0.3, and 25.5°±0.3.

154. The solid form of claim 2, wherein the solid form exhibits the X-ray powder diffraction pattern as shown in FIG. 17.

155. The solid form of any one of claims 143 to 154, wherein the solid form exhibits a differential scanning calorimetry thermogram comprising endothermic peaks at 108.7° C.±5.0 and 195.1° C.±5.0.

156. The solid form of any one of claims 143 to 155, wherein the solid form exhibits the differential scanning calorimetry thermogram as shown in FIG. 18.

157. The solid form of any one of claims 143 to 156, wherein the solid form exhibits less than 7.4%±0.5 weight loss up to 140° C.±10.0 as determined by thermogravimetric analysis.

158. The solid form of any one of claims 143 to 157, wherein the solid form exhibits the thermogravimetric analysis thermogram as shown in FIG. 18.

159. The solid form of any one of claims 143 to 158, wherein the solid form is substantially free of other crystalline or amorphous forms.

160. The solid form of claim 159, wherein the amount of other crystalline or amorphous forms is 5% (w/w) or less.

161. The solid form of any one of claims 143 to 160, wherein the solid form is substantially free of impurities.

162. The solid form of claim 161, wherein the amount of impurities is 2% or less.

163. The solid form of claim 2, wherein the solid form exhibits an X-ray powder diffraction reflection at a 2-theta value of 12.3°±0.3.

164. The solid form of claim 163, wherein the solid form exhibits an X-ray powder diffraction reflection at a 2-theta value of 6.1°±0.3 and 13.4°±0.3.

165. The solid form of any one of claim 163 or 164, wherein the solid form exhibits an X-ray powder diffraction reflection at a 2-theta value of 7.9°±0.3 and 10.5°±0.3.

166. The solid form of any one of claims 163 to 165, wherein the solid form exhibits an X-ray powder diffraction reflection at a 2-theta value of 11.8°±0.3, 25.0°±0.3, and 25.7°±0.3.

167. The solid form of any one of claims 163 to 166, wherein the solid form exhibits an X-ray powder diffraction reflection at a 2-theta value of 18.6°±0.3, 23.0°±0.3, and 25.2°±0.3.

168. The solid form of claim 2, wherein the solid form exhibits at least one X-ray powder diffraction reflection selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

169. The solid form of claim 168, wherein the solid form exhibits at least two X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

170. The solid form of claim 169, wherein the solid form exhibits at least three X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

171. The solid form of claim 170, wherein the solid form exhibits at least four X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

172. The solid form of claim 171, wherein the solid form exhibits at least five X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

173. The solid form of claim 172, wherein the solid form exhibits at least six X-ray powder diffraction reflections selected from 6.1°±0.3, 7.9°±0.3, 10.5°±0.3, 11.8°±0.3, 12.3°±0.3, 13.4°±0.3, 18.6°±0.3, 23.0°±0.3, 25.0°±0.3, 25.2°±0.3, and 25.7°±0.3.

174. The solid form of claim 2, wherein the solid form exhibits the X-ray powder diffraction pattern as shown in FIG. 19.

175. The solid form of any one of claims 163 to 174, wherein the solid form exhibits a differential scanning calorimetry thermogram comprising an endothermic peak at 179.3° C.±5.0.

176. The solid form of any one of claims 163 to 175, wherein the solid form exhibits the differential scanning calorimetry thermogram as shown in FIG. 20.

177. The solid form of any one of claims 163 to 176, wherein the solid form exhibits less than 2.9%±0.5 weight loss up to 245° C.±10.0 as determined by thermogravimetric analysis.

178. The solid form of any one of claims 163 to 177, wherein the solid form exhibits the thermogravimetric analysis thermogram as shown in FIG. 20.

179. The solid form of any one of claims 163 to 178, wherein the solid form is substantially free of other crystalline or amorphous forms.

180. The solid form of claim 179, wherein the amount of other crystalline or amorphous forms is 5% (w/w) or less.

181. The solid form of any one of claims 163 to 180, wherein the solid form is substantially free of impurities.

182. The solid form of claim 181, wherein the amount of impurities is 2% or less.

183. The solid form of claim 1, wherein the solid form is amorphous.

184. The solid form of claim 183, wherein the solid form exhibits the X-ray powder diffraction pattern as shown in FIG. 1.

185. A pharmaceutical composition comprising the solid form of any one of claims 1 to 184 and a pharmaceutically acceptable excipient.

186. A pharmaceutical composition comprising the crystalline form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide as described in any one of claims 3 to 22, and a pharmaceutically acceptable excipient.

187. A pharmaceutical composition comprising the crystalline form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide as described in any one of claims 23 to 42, and a pharmaceutically acceptable excipient.

188. The pharmaceutical composition of claim 186 or 187, further comprising a disintegrating agent.

189. A method of treating cancer in a patient in need thereof, comprising administering to the patient a composition comprising the solid form of any one of claims 1 to 184 and at least one pharmaceutically acceptable excipient.

190. A method of treating cancer in a patient in need thereof, comprising administering to the patient the solid form of any one of claims 1 to 184.

191. A method of treating cancer in a patient in need thereof, comprising administering to the patient the composition of claim 186.

192. A method of treating cancer in a patient in need thereof, comprising administering to the patient the composition of claim 187.

193. A method of treating cancer in a patient in need thereof, comprising administering to the patient the crystalline form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide as described in any one of claims 3 to 22.

194. A method of treating cancer in a patient in need thereof, comprising administering to the patient the crystalline form of 1-((3S,5R)-1-acryloyl-5-(methoxymethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide as described in any one of claims 23 to 42.
